SEQUENCE LISTING (74 sequences)

SEQ ID NO: 1 - DNA, 2756 bp, Homo sapiens
ccgccgggtgcctcgagggcgcgaggccagcccgcctgcccagcccgggaccagcctccccgcgcagcctggcaggtctcctggaggcaaggcgaccttgcttgccctctattgcagaataacaaggggcttagccacaggagttgctggcaagtggaaagaagaacaaatgagtcaatcccgacgtgtcaatcccgacgatagagagctcggaggtgatccacaaatccaagcacccagagatcaattgggatccttggcagatggacatcagtgtcatttactaaccagcaggatggagacgacgcccttgaattctcagaagcagctatcagcgtgtgaagatggagaagattgtcaggaaaacggagttctacagaaggttgttcccaccccaggggacaaagtggagtccgggcaaatatccaatgggtactcagcagttccaagtcctggtgcgggagatgacacacggcactctatcccagcgaccaccaccaccctagtggctgagcttcatcaaggggaacgggagacctggggcaagaaggtggatttccttctctcagtgattggctatgctgtggacctgggcaatgtctggcgcttcccctacatatgttaccagaatggagggggggcattcctcctcccctacaccatcatggccatttttgggggaatcccgctcttttacatggagctcgcactgggacagtaccaccgaaatggatgcatttcaatatggaggaaaatctgcccgattttcaaagggattggttatgccatctgcatcattgccttttacattgcttcctactacaacaccatcatggcctgggcgctatactacctcatctcctccttcacggaccagctgccctggaccagctgcaagaactcctggaacactggcaactgcaccaattacttctccgaggacaacatcacctggaccctccattccacgtcccctgctgaagaattttacacgcgccacgtcctgcagatccaccggtctaaggggctccaggacctggggggcatcagctggcagctggccctctgcatcatgctgatcttcactgttatctacttcagcatctggaaaggcgtcaagacctctggcaaggtggtgtgggtgacagccaccttcccttatatcatcctttctgtcctgctggtgaggggtgccaccctccctggagcctggaggggtgttctcttctacttgaaacccaattggcagaaactcctggagacaggggtgtggatagatgcagccgctcagatcttcttctctcttggtccgggctttggggtcctgctggcttttgctagctacaacaagttcaacaacaactgctaccaagatgccctggtgaccagcgtggtgaactgcatgacgagcttcgtttcgggatttgtcatcttcacagtgctcggttacatggctgagatgaggaatgaagatgtgtctgaggtggccaaagacgcaggtcccagcctcctcttcatcacgtatgcagaagcgatagccaacatgccagcgtccactttctttgccatcatcttctttctgatgttaatcacgctgggcttggacagcacgtttgcaggcttggagggggtgatcacggctgtgctggatgagttcccacacgtctgggccaagcgccgggagcggttcgtgctcgccgtggtcatcacctgcttctttggatccctggtcaccctgacttttggaggggcctacgtggtgaagctgctggaggagtatgccacggggcccgcagtgctcactgtcgcgctgatcgaagcagtcgctgtgtcttggttctatggcatcactcagttctgcagggacgtgaaggaaatgctcggcttcagcccggggtggttctggaggatctgctgggtggccatcagccctctgtttctcctgttcatcatttgcagttttctgatgagcccgccacaactacgacttttccaatataattatccttactggagtatcatcttgggttactgcataggaacctcatctttcatttgcatccccacatatatagcttatcggttgatcatcactccagggacatttaaagagcgtattattaaaagtattaccccagaaacaccaacagaaattccttgtggggacatccgcttgaatgctgtgtaacacactcaccgagaggaaaaaggcttctccacaacctcctcctccagttctgatgaggcacgcctgccttctcccctccaagtgaatgagtttccagctaagcctgatgatggaagggccttctccacagggacacagtctggtgcccagactcaaggcctccagccacttatttccatggattcccctggacatattcccatggtagactgtgacacagctgagctggcctattttggacgtgtgaggatgtggatggaggtgatgaaaaccaccctatcatcagttaggattaggtttagaatcaagtctgtgaaagtctcctgtatcatttcttggtatgatcattggtatctgatatctgtttgcttctaaaggtttcactgttcatgaatacgtaaactgcgtaggagagaacagggatgctatctcgctagccatatattttctgagtagcatatataattttattgctggaatctactagaaccttctaatccatgtgctgctgtggcatcaggaaaggaagatgtaagaagctaaaatgaaaaatagtgtgtccatgcaa

SEQ ID NO: 2 - protein, 630 aa, Homo sapiens
METTPLNSQKQLSACEDGEDCQENGVLQKVVPTPGDKVESGQISNGYSAVPSPGAGDDTRHSIPATTTTLVAELHQGERETWGKKVDFLLSVIGYAVDLGNVWRFPYICYQNGGGAFLLPYTIMAIFGGIPLFYMELALGQYHRNGCISIWRKICPIFKGIGYAICIIAFYIASYYNTIMAWALYYLISSFTDQLPWTSCKNSWNTGNCTNYFSEDNITWTLHSTSPAEEFYTRHVLQIHRSKGLQDLGGISWQLALCIMLIFTVIYFSIWKGVKTSGKVVWVTATFPYIILSVLLVRGATLPGAWRGVLFYLKPNWQKLLETGVWIDAAAQIFFSLGPGFGVLLAFASYNKFNNNCYQDALVTSVVNCMTSFVSGFVIFTVLGYMAEMRNEDVSEVAKDAGPSLLFITYAEAIANMPASTFFAIIFFLMLITLGLDSTFAGLEGVITAVLDEFPHVWAKRRERFVLAVVITCFFGSLVTLTFGGAYVVKLLEEYATGPAVLTVALIEAVAVSWFYGITQFCRDVKEMLGFSPGWFWRICWVAISPLFLLFIICSFLMSPPQLRLFQYNYPYWSIILGYCIGTSSFICIPTYIAYRLIITPGTFKERIIKSITPETPTEIPCGDIRLNAV

SEQ ID NO: 3 - DNA, 23 bp, Artificial Sequence
  Description of Artificial Sequence  Synthetic Primer
gctcaagcaggtgaacaaagaaa

SEQ ID NO: 4 - DNA, 18 bp, Artificial Sequence
  Description of Artificial Sequence  Synthetic Primer
ctgggcagctgggaagag

SEQ ID NO: 5 - DNA, 20 bp, Artificial Sequence
  Description of Artificial Sequence  Synthetic Primer
ggcgttgccgctctgaatgc

SEQ ID NO: 6 - DNA, 23 bp, Artificial Sequence
  Description of Artificial Sequence  Synthetic Primer
gagggactgagctggacaaccac

SEQ ID NO: 7 - DNA, 33 bp, Artificial Sequence
  Description of Artificial Sequence  Synthetic Primer
tgtatgtatttttaccatcagttttgtccagaa

SEQ ID NO: 8 - DNA, 26 bp, Artificial Sequence
  Description of Artificial Sequence  Synthetic Primer
gagagttagctagcaggctcataaat

SEQ ID NO: 9 - DNA, 19 bp, Artificial Sequence
  Description of Artificial Sequence  Synthetic Primer
ttttcctgccacgcactct

SEQ ID NO: 10 - DNA, 25 bp, Artificial Sequence
  Description of Artificial Sequence  Synthetic Primer
gcacaaacctcataagaacctgctt

SEQ ID NO: 11 - DNA, 18 bp, Artificial Sequence
  Description of Artificial Sequence  Synthetic Primer
tggcagtgaccgttccaa

SEQ ID NO: 12 - DNA, 24 bp, Artificial Sequence
  Description of Artificial Sequence  Synthetic Primer
ttgctcaatttgcacaaacctcat

SEQ ID NO: 13 - DNA, 17 bp, Artificial Sequence
  Description of Artificial Sequence  Synthetic Primer
cccagtggaggcacagg

SEQ ID NO: 14 - DNA, 22 bp, Artificial Sequence
  Description of Artificial Sequence  Synthetic Primer
gagtgtgcaggttactgatgct

SEQ ID NO: 15 - DNA, 17 bp, Artificial Sequence
  Description of Artificial Sequence  Synthetic Primer
ggaggcaaggcgacctt

SEQ ID NO: 16 - DNA, 23 bp, Artificial Sequence
  Description of Artificial Sequence  Synthetic Primer
ctgtggctaagccccttgttatt

SEQ ID NO: 17 - DNA, 25 bp, Artificial Sequence
  Description of Artificial Sequence  Synthetic Primer
gtcatttactaaccagcaggatgga

SEQ ID NO: 18 - DNA, 21 bp, Artificial Sequence
  Description of Artificial Sequence  Synthetic Primer
cgctgatagctgcttctgaga

SEQ ID NO: 19 - DNA, 22 bp, Artificial Sequence
  Description of Artificial Sequence  Synthetic Primer
gggtactcagcagttccaagtc

SEQ ID NO: 20 - DNA, 19 bp, Artificial Sequence
  Description of Artificial Sequence  Synthetic Primer
gggatagagtgccgtgtgt

SEQ ID NO: 21 - DNA, 25 bp, Artificial Sequence
  Description of Artificial Sequence  Synthetic Primer
tggatttccttctctcagtgattgg

SEQ ID NO: 22 - DNA, 25 bp, Artificial Sequence
  Description of Artificial Sequence  Synthetic Primer
tcatgttcctagtcttacgccagtg

SEQ ID NO: 23 - DNA, 27 bp, Artificial Sequence
  Description of Artificial Sequence  Synthetic Primer
gctatactacctcatctcctccttcac

SEQ ID NO: 24 - DNA, 19 bp, Artificial Sequence
  Description of Artificial Sequence  Synthetic Primer
tggtgcagttgccagtgtt

SEQ ID NO: 25 - DNA, 20 bp, Artificial Sequence
  Description of Artificial Sequence  Synthetic Primer
tcctggaacactggcaactg

SEQ ID NO: 26 - DNA, 21 bp, Artificial Sequence
  Description of Artificial Sequence  Synthetic Primer
gaatggagggtccaggtgatg

SEQ ID NO: 27 - DNA, 24 bp, Artificial Sequence
  Description of Artificial Sequence  Synthetic Primer
actccaagggttgtgatctttctg

SEQ ID NO: 28 - DNA, 25 bp, Artificial Sequence
  Description of Artificial Sequence  Synthetic Primer
gggtgaatggatgtcagtgtctttt

SEQ ID NO: 29 - DNA, 24 bp, Artificial Sequence
  Description of Artificial Sequence  Synthetic Primer
gtgacagccaccttcccttatatc

SEQ ID NO: 30 - DNA, 17 bp, Artificial Sequence
  Description of Artificial Sequence  Synthetic Primer
gtggcacccctcaccag

SEQ ID NO: 31 - DNA, 22 bp, Artificial Sequence
  Description of Artificial Sequence  Synthetic Primer
agccgctcagatcttcttctct

SEQ ID NO: 32 - DNA, 26 bp, Artificial Sequence
  Description of Artificial Sequence  Synthetic Primer
ttgaacttgttgtagctagcaaaagc

SEQ ID NO: 33 - DNA, 17 bp, Artificial Sequence
  Description of Artificial Sequence  Synthetic Primer
cggccccttgggttttc

SEQ ID NO: 34 - DNA, 21 bp, Artificial Sequence
  Description of Artificial Sequence  Synthetic Primer
gaagctcgtcatgcagttcac

SEQ ID NO: 35 - DNA, 21 bp, Artificial Sequence
  Description of Artificial Sequence  Synthetic Primer
gcagaagcgatagccaacatg

SEQ ID NO: 36 - DNA, 22 bp, Artificial Sequence
  Description of Artificial Sequence  Synthetic Primer
caagcccagcgtgattaacatc

SEQ ID NO: 37 - DNA, 27 bp, Artificial Sequence
  Description of Artificial Sequence  Synthetic Primer
tccccacatatatagcttatcggttga

SEQ ID NO: 38 - DNA, 29 bp, Artificial Sequence
  Description of Artificial Sequence  Synthetic Primer
caaaacaattagtagtctgaacacacaca

SEQ ID NO: 39 - DNA, 32 bp, Artificial Sequence
  Description of Artificial Sequence  Synthetic Primer
gcgtattattaaaagtattaccccagaaacac

SEQ ID NO: 40 - DNA, 21 bp, Artificial Sequence
  Description of Artificial Sequence  Synthetic Primer
cacagcattcaagcggatgtc

SEQ ID NO: 41 - DNA, 20 bp, Artificial Sequence
  Description of Artificial Sequence  Synthetic Primer
aactattgctatgcggtgat

SEQ ID NO: 42 - DNA, 15 bp, Artificial Sequence
  Description of Artificial Sequence  Synthetic Primer
ttgctgtgcggtgat

SEQ ID NO: 43 - DNA, 16 bp, Artificial Sequence
  Description of Artificial Sequence  Synthetic Primer
cattgaccaggttcac

SEQ ID NO: 44 - DNA, 16 bp, Artificial Sequence
  Description of Artificial Sequence  Synthetic Primer
cattgacctggttcac

SEQ ID NO: 45 - DNA, 15 bp, Artificial Sequence
  Description of Artificial Sequence  Synthetic Primer
accgttccaatatgg

SEQ ID NO: 46 - DNA, 14 bp, Artificial Sequence
  Description of Artificial Sequence  Synthetic Primer
ccgttccaacatgg

SEQ ID NO: 47 - DNA, 18 bp, Artificial Sequence
  Description of Artificial Sequence  Synthetic Primer
ctgcttctcactcatcca

SEQ ID NO: 48 - DNA, 17 bp, Artificial Sequence
  Description of Artificial Sequence  Synthetic Primer
tgcttctcactcaacca

SEQ ID NO: 49 - DNA, 15 bp, Artificial Sequence
  Description of Artificial Sequence  Synthetic Primer
tggttggtgtcgccg

SEQ ID NO: 50 - DNA, 15 bp, Artificial Sequence
  Description of Artificial Sequence  Synthetic Primer
tggttggtttcgccg

SEQ ID NO: 51 - DNA, 17 bp, Artificial Sequence
  Description of Artificial Sequence  Synthetic Primer
cttgccctctattgcag

SEQ ID NO: 52 - DNA, 16 bp, Artificial Sequence
  Description of Artificial Sequence  Synthetic Primer
ttgccctctcttgcag

SEQ ID NO: 53 - DNA, 16 bp, Artificial Sequence
  Description of Artificial Sequence  Synthetic Primer
attcaagggcgtcgtc

SEQ ID NO: 54 - DNA, 13 bp, Artificial Sequence
  Description of Artificial Sequence  Synthetic Primer
caagggcgccgtc

SEQ ID NO: 55 - DNA, 14 bp, Artificial Sequence
  Description of Artificial Sequence  Synthetic Primer
ctggtgcgggagat

SEQ ID NO: 56 - DNA, 14 bp, Artificial Sequence
  Description of Artificial Sequence  Synthetic Primer
ctggtgcggcagat

SEQ ID NO: 57 - DNA, 17 bp, Artificial Sequence
  Description of Artificial Sequence  Synthetic Primer
ccaggagttcttgcagc

SEQ ID NO: 58 - DNA, 16 bp, Artificial Sequence
  Description of Artificial Sequence  Synthetic Primer
caggagttgttgcagc

SEQ ID NO: 59 - DNA, 20 bp, Artificial Sequence
  Description of Artificial Sequence  Synthetic Primer
aattacttctccgaggacaa

SEQ ID NO: 60 - DNA, 20 bp, Artificial Sequence
  Description of Artificial Sequence  Synthetic Primer
aattacttctccaaggacaa

SEQ ID NO: 61 - DNA, 16 bp, Artificial Sequence
  Description of Artificial Sequence  Synthetic Primer
accacctcaccctcct

SEQ ID NO: 62 - DNA, 14 bp, Artificial Sequence
  Description of Artificial Sequence  Synthetic Primer
cacctcgccctcct

SEQ ID NO: 63 - DNA, 15 bp, Artificial Sequence
  Description of Artificial Sequence  Synthetic Primer
caggacagaaaggat

SEQ ID NO: 64 - DNA, 15 bp, Artificial Sequence
  Description of Artificial Sequence  Synthetic Primer
caggacaaaaaggat

SEQ ID NO: 65 - DNA, 15 bp, Artificial Sequence
  Description of Artificial Sequence  Synthetic Primer
caaagcccggaccaa

SEQ ID NO: 66 - DNA, 15 bp, Artificial Sequence
  Description of Artificial Sequence  Synthetic Primer
caaagcccagaccra

SEQ ID NO: 67 - DNA, 18 bp, Artificial Sequence
  Description of Artificial Sequence  Synthetic Primer
ccagagatgccctggtga

SEQ ID NO: 68 - DNA, 17 bp, Artificial Sequence
  Description of Artificial Sequence  Synthetic Primer
cagagatgccatggtga

SEQ ID NO: 69 - DNA, 18 bp, Artificial Sequence
  Description of Artificial Sequence  Synthetic Primer
tttctttgccatcatctt

SEQ ID NO: 70 - DNA, 16 bp, Artificial Sequence
  Description of Artificial Sequence  Synthetic Primer
tctttgccgtcatctt

SEQ ID NO: 71 - DNA, 17 bp, Artificial Sequence
  Description of Artificial Sequence  Synthetic Primer
cacgtacctctttaaat

SEQ ID NO: 72 - DNA, 16 bp, Artificial Sequence
  Description of Artificial Sequence  Synthetic Primer
acgtacctcgttaaat

SEQ ID NO: 73 - DNA, 14 bp, Artificial Sequence
  Description of Artificial Sequence  Synthetic Primer
cacaaggaatttct

SEQ ID NO: 74 - DNA, 14 bp, Artificial Sequence
  Description of Artificial Sequence  Synthetic Primer
cacaagaaatttct

(19) United States
(12) Patent Application Publication
(10) Pub. No.: US 2007/0037194 A1
(43) Pub. Date: Feb. 15, 2007

(54) Allelic variation in the serotonin transporter (SERT) as an indicator of autism

(75) Inventors: James Sutcliffe, Nashville, TN (US); Randy Blakely, Brentwood, TN (US); Harish Prasad, Nashville, TN (US); Ryan Delahanty, Nashville, TN (US)

Correspondence Address: FULBRIGHT & JAWORSKI L.L.P., 600 CONGRESS AVE., SUITE 2400, AUSTIN, TX, 78701, US

(73) Assignee: VANDERBILT UNIVERSITY

(21) Appl. No.: 11/501,663

(22) Filed: Aug. 9, 2006

Related U.S. Application Data
(60) Provisional application No. 60/706,634, filed on Aug. 9, 2005.

(51) Int. Cl. C12Q 1/68

(52) U.S. Cl. 435/6

(57) ABSTRACT

The present invention describes allelic variations in the SERT gene that are linked with the development of autism.

This application claims benefit of priority to U.S. Provisional Application Ser. No. 60/706,634, filed Aug. 9, 2005, the entire contents of which are hereby incorporated by reference.

The government owns rights in the present invention pursuant to grant numbers MH61009, NS026630 and DA07390 from the National Institutes of Health.

### BACKGROUND OF THE INVENTION

1. Field of the Invention

The present invention relates generally to the fields of neurobiology and genetics and pharmacology. More particularly, it concerns assessing allelic variation in the SERT gene as a method for identifying subjects having or at risk of developing autism.

2. Description of Related Art

In 1943, Leo Kanner published a series of eleven case reports of children with a condition he termed “infantile autism” or “autistic disturbances of affective contact” (Kanner, 1943). Autism is now recognized as a spectrum of phenotypes spanning a range of clinical severity but fundamentally representing deficits in three domains: (1) development and use of language, (2) development of social relationships and interactions with family and peers, and (3) patterns of repetitive behaviors, restricted interests and activities, and a strong desire to maintain “sameness” in their environment and daily routines (reviewed in Folstein and Rosen-Sheidley, 2001). Pharmacotherapies of disruptive and aggressive behaviors involve using antipsychotic medications and selective serotonin reuptake inhibitors (SSRIs) for anxiety, depression and repetitive behaviors (Cook and Leventhal, 1996; Hollander et al., 2005).

Of interest regarding the partial efficacy of SSRIs are findings of elevated levels of platelet serotonin (5-hydroxytryptamine; 5-HT) in ˜20-25% of affected individuals (Schain and Freedman, 1961) and correlated with levels in first degree relatives (Cook et al., 1993). Other findings supporting 5-HT involvement in autism are reviewed elsewhere (Cook and Leventhal, 1996; Veenstra-VanderWeele et al., 2000). Recent research also has shown that polymorphisms in the promoters of SERT's are a risk factor for susceptibility to depression (Neumeister et al., 2002). Other studies have also shown the association of variants of SERT's to other disorders. For example, association for allele 12 of the variable number tandem repeat (VNTR) in the second intron of the SERT gene and schizophrenic disorders has been shown (Tsai et al., 2002).

Since the first twin study in autism by Folstein and Rutter in 1977 (Folstein and Rutter, 1977), evidence has mounted to support a predominantly genetic etiology in autism (reviewed in Folstein and Rosen-Sheidley, 2001). The prevalence of narrowly defined autism is ˜ 1/1000 (Chakrabarti and Fombonne, 2001), and inclusion of the broader spectrum increases this rate to ˜ 1/300- 1/500 (Fombonne, 2003; Yeargin-Allsopp et al., 2003). Males are affected more often, with a male:female ratio of 4:1. Twin data show that monozygotic (MZ) twins have an average concordance of ˜60-70% for classic autism and up to 90% when milder language and social deficits seen in the broader phenotype are considered. This contrasts with dizygotic concordance rates, shown to be 0-10% depending on study. Sibling recurrence risk in narrowly defined autism is ˜6-8% (Jones and Szatmari, 1988; Ritvo et al., 1989). Modeling the above data have led to estimates of between ˜5 to 15 genes contributing to genetic risk, possibly involving epistasis (Pickles et al., 1995), and locus heterogeneity (Risch et al., 1999). The data may best be explained by oligogenic inheritance, with different families possessing varying constellations of risk alleles (Folstein and Rosen-Sheidley 2001). A genetic heterogeneity framework has important implications for the clinical variability observed in autism, in that specific risk loci, or distinct alleles at a given locus, are likely to influence phenotype differently.

Several groups have undertaken family-based genetic studies to (1) identify regions of the genome commonly inherited by affected family members in multiplex-family samples (i.e., genomic linkage screens) and/or (2) test specific loci for evidence of common alleles conferring genetic risk based on tests of allelic association (reviewed in Folstein and Rosen-Sheidley, 2001; Veenstra-Vanderweele et al., 2004). Chromosomal intervals identified in linkage studies using either a categorical diagnosis or indexing on specific traits include 7q, 2q, and 17q (IMGSAC, 1998; Ashley-Koch et al., 1999; C L S A et al., 1999; Philippe et al., 1999; Risch et al., 1999; Auranen et al., 2000; Buxbaum et al., 2001; CLSA, 2001; International Molecular Genetic Study of Autism, 2001; International Molecular Genetic Study of Autism Consortium, 2001; Liu et al., 2001; Shao et al., 2002a; Shao et al., 2002b; Yonan et al., 2003; Buxbaum et al., 2004; McCauley et al., 2004; Stone et al., 2004; Cantor et al., 2005; McCauley et al., 2005). Regarding 17q, second-stage genomic screens using different samples (International Molecular Genetic Study of Autism Consortium, 2001; Yonan et al., 2003) detected highly suggestive or significant linkage at or near the serotonin transporter (SERT) locus (SLC6A4), and evidence for a sex-restricted pattern of genetic effects (Stone et al., 2004). Our own reports of linkage to this region, using family datasets containing partial overlap with the Autism Genetics Resource Exchange (AGRE) sample (Yonan et al., 2003), lend further support to involvement of this region (McCauley et al., 2004; McCauley et al., 2005).

Studies of allelic association at SLC6A4 in autism have focused primarily on a functional, insertion-deletion polymorphism in the promoter (5-HTTLPR) or a variable number tandem repeat (VNTR) marker in intron 2. Results of these studies are inconsistent, with associations shown to either the short (S) or long (L) alleles, or absent association (Cook et al., 1997; Klauck et al., 1997; Maestrini et al., 1999; Persico et al., 2000; Tordjman et al., 2001; Yirmiya et al., 2001). Further studies have reported more comprehensive analyses of common alleles and haplotypes spanning the locus, including multiple single nucleotide polymorphisms (SNPs) (Kim et al., 2002; Conroy et al., 2004; McCauley et al., 2004). These studies find at least nominal association to the S allele at 5-HTTLPR and other markers or haplotypes. The inventors' own study of multiplex families (McCauley et al., 2004) also showed a very suggestive linkage, which cannot be explained by the modest association of HTTLPR and rs140700 at SLC6A4. The two possible explanations for these results are (1) that SLC6A4 is not the risk locus accounting for linkage or (2) that multiple different alleles at SLC6A4 contribute to genetic risk independently. Regardless, there remains a need for better information regarding genetic linkage to a predisposition to autism.

### SUMMARY OF THE INVENTION

Thus, present invention provides a method of identifying a subject having or at risk of developing autism, an austism spectrum disorder or an associated disorder comprising assessing the expression or mutation of a gene located at SLC6A4. The subject may or may not exhibit one or more clinical symptoms of autism. The subject may or may not have previously been diagnosed with autism, or have a family member that has previously been diagnosed with autism. The subject may be male or female, and the method may further comprise making at treatment decision based on the assessing.

Assessing may comprise measuring the expression level of SERT protein, determining the structure of SERT protein, or measuring the expression level or structure of a SLC6A4 transcript (e.g., Northern blot or quantitative RT-PCR of SLC6A4), determining the structure of a SLC6A4 gene, determining the structure of a SLC6A4 coding region, determining the structure of a SLC6A4 non-coding region (such as a promoter, intron or 3′ non-coding region). Specific assessing techniques comprise sequencing, primer extension, restriction digestion, SNP-specific oligonucleotide hybridization, or DNAse protection.

The mutation being assessed may comprise a change in SLC6A4 exon 1b, exon 2, exon 9, exon, 10, exon 12, exon 13, exon 14, intron 1a, intron 1b, intron 6, intron 7, intron 8, or the SLC6A4 promoter. The subject may exhibit one or more change selected from Gly56Ala (462G→C), Ile425Leu (1568A462G→C), Phe465Leu (1688T462G→C) and Leu550Val 1943G462G→C), two or more changes selected from Gly56Ala, Ile425Leu, Phe465Leu and Leu550Val, three or more changes selected from Gly56Ala, Ile425Leu, Phe465Leu and Leu550Val, or all of these changes. Other changes include nucleotide alterations selected from the following: 147C→A, 2517A→G, 15622G→A, 14519A→T, 14289A→C, 13912T→C, 13754C→T, IVS1a+20C→T, IVS1a+133G→A, IVS1a-47G→C, IVS1a-25G→A, IVS6-44G→C, ISV7+83C→T and ISV8-33C→T.

In another embodiment, there is provided a method of identifying a subject having or at risk of developing autism comprising assessing the subject's SERT activity. The method may further comprise obtaining a tissue sample from said subject. The subject may exhibit one or more clinical symptoms of autism, or may not. The subject may or may not have previously been diagnosed with autism, or have a family member that has previously been diagnosed with autism. The subject may be male or female. The method may further comprising making at treatment decision based on the assessing.

In yet another embodiment, there is provided a nucleic acid primer for amplification of a SLC6A4 gene at a position selected from the group consisting 425, 465 and 550 of SEQ ID NO:1. In still yet another embodiment, there is provided a nucleic acid probe that selectively hybridizes to a SLC6A4 gene encoding 425Leu, 465Leu or 550Val of SEQ ID NO:2. In a further embodiment, there is provided an antibody that binds to a SERT protein having 425Leu, but that does not bind to a SERT protein have 42511e. In still a further embodiment, there is provided an antibody that binds to a SERT protein having 465Leu, but that does not bind to a SERT protein have 425Phe. In still yet a further embodiment, there is provide an antibody that binds to a SERT protein having 550Val, but that does not bind to a SERT protein have 550Leu.

Another embodiment comprises a method of screening for an agent that can modulate one or more symptoms of autism, an autism spectrum disorder or an associated disorder comprising (a) contacting a cell that expresses SERT with said agent; (b) measuring SERT activity; and (c) comparing the SERT activity observed in (b) with that seen in the absence of said agent, whereby a difference in the activity observed in (b) and (c) indicates that said agent modulates one or more symptoms of autism. The agent may inhibit or increase SERT activity, such as an antisense SERT nucleic acid, SERT siRNA or SERT-binding Ab (antagonist), or SERT-encoding expression construct (agonist).

It is contemplated that any method or composition described herein can be implemented with respect to any other method or composition described herein. The use of the word, “a” or “an” when used with the term “comprising” in the specification and/or claims may mean “one”, “one or more,” “at least one,” or “one or more than one.” Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-F. Male-biased linkage of autism and novel coding variants at the 17q11.2 SLC6A4 locus. (FIG. 1A) Male-biased linkage of autism to 17q11.2. Multipoint linkage analysis on chromosome 17 is shown for the overall 340-family dataset (black line), 202 families containing only affected males (MO; blue line), or the remaining 138 families containing one or more affected females (FC; red line). Multipoint HLOD scores were calculated under a recessive model and plotted as a function of marker position in centimorgans (cM) along chromosome 17. (FIGS. 1B-D) Sequence detection of novel nonsynonymous SLC6A4 variants in autism families. Sequence-based detection is shown for each of the three novel coding variants along with corresponding pedigrees (FIG. 1B) Ile425Leu, (FIG. 1C) Phe465Leu, (FIG. 1D) Leu550Val. Filled circles or squares reflect individuals with an autism diagnosis, open circles or squares reflect individuals without autism, and allele carriers without autism are indicated by small filled circles or squares within the larger pedigree symbol. Electropherogram data is shown in either sense (FIG. 1B) or antisense (FIG. 1A) and (FIG. 1C) orientations, respectively, with corresponding coding sequence. Antisense sequences (FIGS. 1A and 1C) indicate the reversed orientation of amino acid codons, represented by lines across each three-base sequence. Variant amino acids are shown in red, and corresponding heterozygous sequence changes are indicated by an arrow. Individual numbers in the respective pedigrees correspond to numbers within each of the sequence frames. (FIG. 1E) A schematic representation of the 5-HT transporter is shown. Amino acid substitutions are indicated by location within transmembrane or cytoplasmic domains. (FIG. 1F) Dosage dependent elevated 5-HT transporter activity of Ala56-encoded hSERT in native lymphoblastoid cells. Lymphocytes genotype-matched at 5HTTLPR (L/L) and the intron 2 VNTR (10/10) and bearing Gly/Gly, Gly/Ala or Ala/Ala-encoding genotypes at residue 56 were assayed for [³H]5-HT transport activity as described (see Methods). Three independent experiments were performed in triplicate using each line and the combined basal uptake data plotted.

FIGS. 2A-D. ClustalW alignment of hSERT amino acid sequence against other species of SERT and other biogenic amine transporters. (FIG. 2A) Gly56 is conserved in mammalian species of SERT. (FIG. 2B) Ile425 is conserved in all SERT proteins from human to Drosophila. (FIG. 2C) Phe465 is conserved in all species shown for all monoamine transporters, and is also conserved in glycine, α-aminobutyric acid and creatine transporters. (FIG. 2D) Leu550 is conserved in all monoamine transporters from human to Drosophila.

FIGS. 3A-B. SERT is refractory to regulation through PKG and p38 MAP kinase signaling pathways. (FIG. 3A) SERT Ala56 lacks sensitivity to 8BrcGMP. Homozygous Ala56 cells (10⁶/tube) were preincubated for various times at 37° C. with 10 μM 8BrcGMP prior to [³H]5-HT transport assays. (FIG. 3B) SERT Ala56 lacks sensitivity to the p38 MAPK activator anisomycin. 8BrcGMP effects on Gly56 SERT are completely blocked by coincubation with the PKG inhibitor H8 (10 μM) whereas the p38 MAPK inhibitor SB203580 (1 μM) prevented anisomycin stimulation (data not shown) Data plotted represent mean data+/−SD (n=3) for a single cell line of each genotype assayed in parallel. Findings were replicated with identical results in an additional line for each genotype.

FIGS. 4A-D. Altered basal 5-HT transport activity and regulation associated with the Gly56Ala variant. [³H]5-HT (5HT) and [³H]L-glutamate (Glu) transport activities were defined as described in Methods. (FIG. 4A) 5-HT transport activity of EBV-transformed lymphocyte cell lines by genotype (G=Gly56, A=Alanine56). (FIG. 4B) Lglutamate transport activity of the lines tested in A. (FIG. 4C) Normalized 5-HT transport activity, with an individual cell lines 5-HT transport activity divided by its L-glutamate transport activity. (FIG. 4D) Average by genotype of the values in C. * denotes p<0.05 (Student's t-test) compared against Gly56 cell.

FIG. 5. Increase 5HT Transport Associated with Autism Mutations in Transfected HeLa Cells. *, P<0.05, ANOVA+posthoc Dunnett tests.

FIG. 6. Autism SERT Mutations Differentially Alter Transporter Surface Expression. *, P<0.05, ANOVA+posthoc Dunnett tests.

FIG. 7. Influence of SERT Coding Variants on 5HT and Glutamate Transport Assessed in Genotyped Lymphocytes.

### DESCRIPTION OF ILLUSTRATIVE EMBODIMENTS

I. The Present Invention

Serotonin and the serotonin transporter mediate diverse aspects of neuronal signaling and are involved in the pathology of a number of nervous system related disorders. The link between SERT and autism has been explored, but remains inconclusive (Cook et al., 1997; Klauck et al., 1997; Maestrini et al., 1999; Persico et al., 2000; Tordjman et al., 2001; Yirmiya et al., 2001; Kim et al., 2002; Conroy et al., 2004; McCauley et al., 2004; McCauley et al., 2004). The inventors now report further investigation of chromosome 17 linkage in autism families and an in depth analysis of the SLC6A4 gene, testing the hypothesis that allelic heterogeneity may account for the genetic liability to autism. Using a large sample of multiplex families, the inventors have now shown that SLC6A4 exhibits strong evidence of linkage to autism, driven by allele sharing in males. Multiple coding and non-coding variants are preferentially transmitted to affected individuals, and identify significant correlations with increased rigid-compulsive behaviors, indicating that SLC6A4 is a likely susceptibility locus in autism, where allelic heterogeneity supports disease risk.

The inventors have discovered three new highly-conserved coding variants in families strongly contributing to linkage in this region. Two of the three coding variants (Ile425Leu and Leu550Val), a 56Ala homozygous proband, and a novel intron 6 SNP (IVS6-44G>C) were identified in 24 probands (8.3% allele frequency) from families with the highest family-specific LOD scores. These two new alleles segregate to all affected individuals in their respective families and thus are associated with disease. These findings spawned further discovery efforts leading to the identification of a total of 13 novel SNPs, including the Phe465Leu nonsynonymous variant. The family with this latter variant is of interest given that the mother carried, but did not transmit, the 56Ala allele. She did transmit a rare genomic variant (hCV11414114, Table 2) to all affected children. The study by Glatt and colleagues (Glatt et al., 2001) did not detect the three novel coding alleles in the 450 individuals (900 chromosomes) sequenced, indicating these alleles are rare and have a frequency of less than 1/900 or 0.011%.

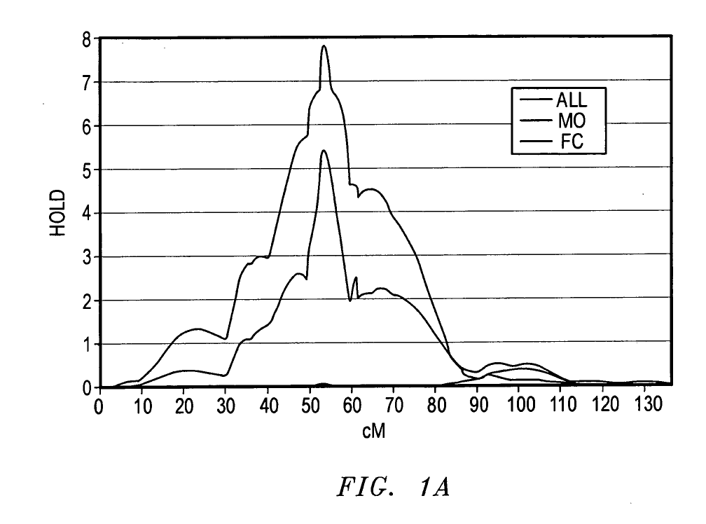

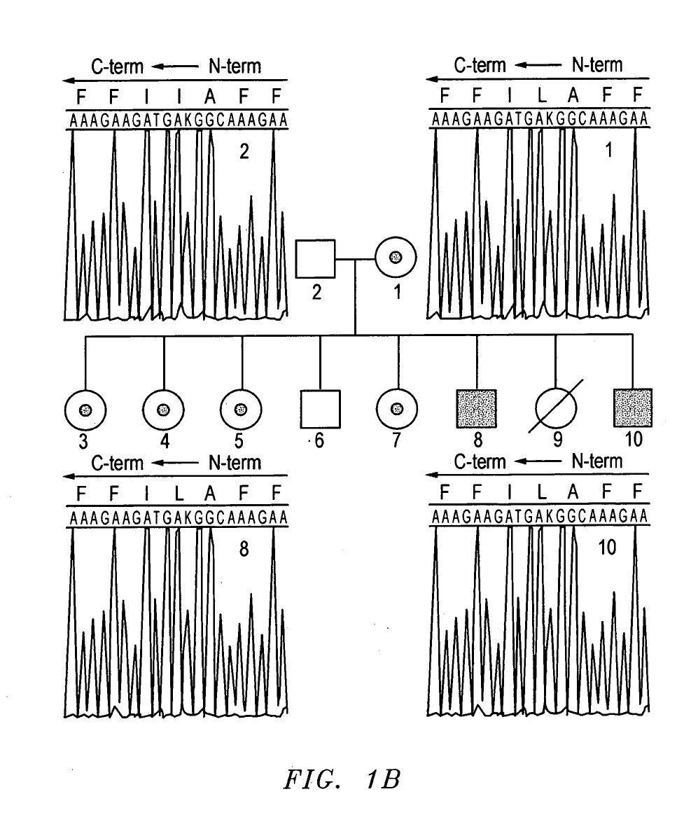

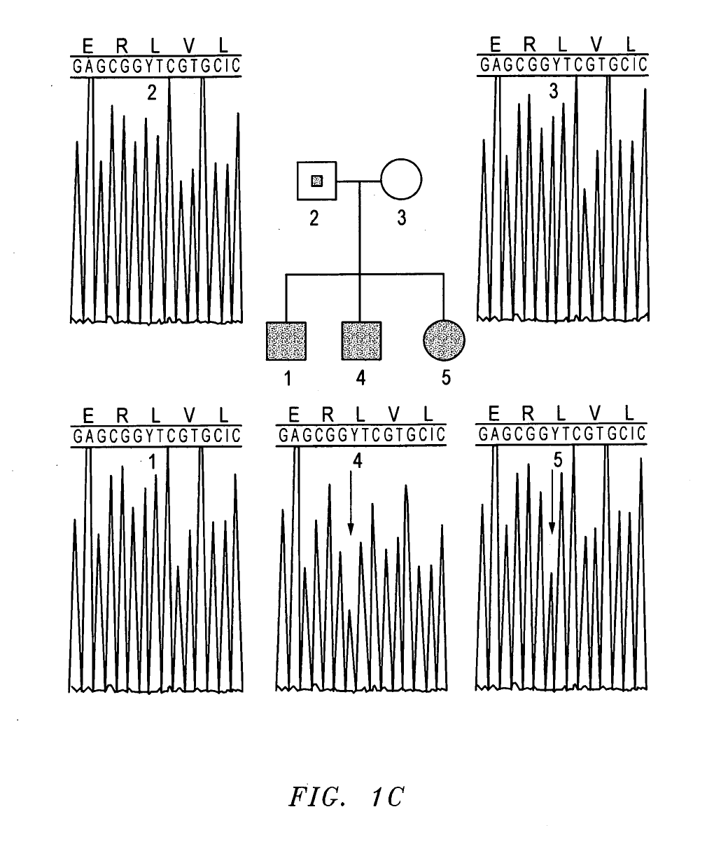

Cross-species conservation of novel coding variant residues and their identification in linked families suggests an increased likelihood of altered functionality for the variant transporter, although future study will be required to fully elaborate this premise. All three new substitutions occur within transmembrane domains (FIG. 1E), and the 425Leu allele affects the identical residue and nucleotide as the Ile425Val mutant in the pedigrees described by Ozaki and colleagues, segregating Asperger syndrome, obsessive-compulsive disorder (OCD) and other psychiatric phenotypes (Ozaki et al., 2003). The Ile425Leu variant does, therefore, have an a priori increased likelihood of functional effect based on prior precedent from disease-association and subsequent functional characterization of the Ile425Val substitution showing ˜two-fold elevated basal activity in transfection studies (Kilic et al., 2003) and Prasad et al., submitted.

The Gly56Ala substitution shows a very suggestive increase in minor allele frequency (MAF) to 2.3% in the 120 linked families, compared to the 1.1% seen in the remaining families. A 2.3% allele frequency represents a noticeable increase over the single non-clinical reference study (Glatt et al., 2001), showing 4 of 900 chromosomes (and no homozygotes) carrying a 56Ala allele (0.44%). HWE would dictate a frequency of homozygous individuals to be ˜ 1/2000 in unrelated individuals (or ˜ 1/5000 based on a 0.44% frequency in the Glatt et al. comparison sample). Therefore, the finding here of three homozygotes and two additional (unrelated) instances of dual heterozygous couples is highly unlikely by chance (P≈0). The findings of (1) an apparent five-fold increase in 56Ala allele frequency in the linked families, with a trend towards overtransmission, (2) a substantial deviation from HWE, and (3) a male-biased trend towards autism affection in the presence of this allele compared to females, indicates a role for the 56Ala allele as a genetic risk factor in autism. The infrequency of the allele, however, makes this more difficult to quantitate. Larger autism population studies are indicated.

Equally important are functional data demonstrating that mutant SERT proteins display elevated basal activity and insensitivity to regulation, in particular, through PKG and p38 MAPK signaling pathways in a native cell system. Importantly, downregulation in response to PKC activating phorbol esters was equivalent between Gly56 and Ala56 lines. The inventors do not believe that PKG/p38 MAPK regulatory insensitivity derives directly from altered basal activities, as the inventors have demonstrated transport regulation through these pathways with other variants differing up to 10-fold in activity (Prasad et al., submitted), although over-expression in transfected cells can blunt regulation (Zhu and Blakely, unpublished findings). Elevated basal 5-HT uptake is also not due to enhanced transcription as RealTime™ PCR analysis revealed equal SERT mRNA levels in the Gly56 and Ala56 lines (data not shown). Elevated basal activity of the Ala56-encoded transporter is intriguing in light of a similar effect with 425Val-encoded SERT in patients with Asperger and OCD.

Phenotypically, the three new variants and Gly56Ala are significantly associated with increased rigid-compulsive behaviors. These include (1) stereotyped utterances, (2) unusual preoccupations, (3) compulsions/rituals, (4) resistance to trivial changes in the environment, and (5) unusual attachment to objects (Tadevosyan-Leyfer et al., 2003). This is important for several reasons. The inventors previous study of linkage in a smaller sample demonstrated a significant increase in linkage at ˜53 cM when the same rigid-compulsive trait was used to stratify the dataset (McCauley et al., 2004). It is consistent with the finding of Asperger syndrome, OCD and other traits in families segregating the functionally-abnormal (Prasad et al., submitted) Ile425Val substitution (Ozaki et al., 2003). Obsessive-compulsive-type traits and clinical OCD are seen more frequently in families with autism than in the general population (Bolton et al., 1998). Repetitive behaviors and associated anxiety in autism, as well as OCD, are often effectively treated with SSRIs, targeting the SERT protein (Hollander et al., 2005). While other phenotypic findings associated with specific variants were variable across families with all four variants, increased severity for the rigid-compulsive domain was a consistent finding for these variants. Given the magnitude of the observed genetic effect, the possibility exists that our sample may be enriched by chance or selection methods for phenotypic traits (or regional alleles) that bias in favor of an effect at this locus.

The functionality of promoter and other noncoding variants is unknown, however, the segregation of these multiple variants in aggregate, in addition to the coding variants, provides additional genetic evidence for an allelic heterogeneity framework for disease risk involving SLC6A4. Several previously documented variants (rs2020932, hCV11414117, and hCV11414114) were found in multiple families each. These heterogeneous variants largely arose on independent haplotypic backgrounds, indicating they are rare independent events, and not resulting from an effect of some common genetic background. The clustering of collectively associated rare genomic variants in the promoter and near the 5′ end of the gene raises the possibility of transcriptional effects at SLC6A4. Intronic or noncoding transcribed variants may, if risk factors with biological relevance, affect transcription, transcript stability or RNA splicing (Pagani and Baralle, 2004). An expression-based mechanism for potentially diseaserelated 5′ variants is consistent with association of the HTTLPR marker, since the insertion/deletion variant exhibits differential transcription. Despite a relatively conservative analysis of transmission by eliminating the subject from each family in whom the variant was discovered, association data in the presence of linkage must be interpreted cautiously. The analysis of transmissions in families with an a priori expectation of variable allele-sharing would inflate any measure of allelic association, and the TDT analysis of multiplex families in this unusual context would yield a measure of linkage (Spielman and Ewens, 1996). Nevertheless, the context in which these observations are made supports the significance of association in the aggregate of these multiple variants. The modest association at 5-HTTLPR and the intron 5 SNP rs140700 in the current MO sample is not only consistent with, but supportive of, this proposed risk framework. Other common alleles are not associated with autism, while the heterogeneous rare alleles described in this report are. Thus, the most parsimonious model involves multiple different risk alleles (including 5-HTTLPR) acting in different families to collectively account for the observed linkage.

The import of the data reported here is summarized by the constellation of findings including: (1) three novel highly conserved coding variants, one of which affects a residue (Ile425) with known phenotypic and functional relevance; (2) the dysfunctional properties of SERT encoded with the various mutations; (3) stark deviation from HWE and increased frequency in linked families; (4) a phenotypic correlation between coding variants and increased rigid-compulsive behaviors and (5) the aggregate association shown by heterogeneous promoter, 5′ and intragenic noncoding variants. These data collectively support the premise that SLC6A4 is a susceptibility locus for autism spectrum disorders. These findings must be examined in a larger independent autism family populations with similar phenotypic representation to determine their ultimate significance. Given the linkage reported at SLC6A4 by IMGSAC (as well as AGRE) and incomplete screening in the current sample (only one affected individual per family was screened for exon and promoter variants), additional coding and noncoding variants will likely be discovered at this locus. As they stand now, the findings provide compelling evidence that the SLC6A4 locus is a bona fide autism susceptibility gene, with variants predisposing to rigid compulsive traits.

II. Serotonin Transporter (SERT)

Selective antagonism of serotonin (5-hydroxytryptamine, 5HT) and noradrenaline (NA) transport by antidepressants is a key element to our current understanding of human behavioral disorders (Ashton, 1987). The serotoninergic system modulates numerous behavioral and physiological functions and has been associated with control of mood, emotion, sleep and appetite. Synaptic serotonin (SE), also called 5-hydroxytryptamine or 5HT, concentration is controlled by the serotonin transporter (SERT) which is involved in reuptake of serotonin into the pre-synaptic terminal. In several studies, 5HT uptake and/or transport sites have been found to be reduced in platelets of patients suffering from depression and reduced in post-mortem brain samples of depressed patients and suicide victims (Meltzer et al., 1981; Suranyi-Cadotte et al., 1985; Briley et al., 1993; Paul et al., 1981; Perry et al., 1983). The cloning of the human SERT protein by Ramamoorthy et al. (1993), shows that human SERT is encoded by a single gene that is localized to chromosome 17q11.1-17q12 and encodes for a 630-amino acid protein. The hSERT is a Na⁺- and Cl⁻ coupled serotonin transporter and has been found to be expressed on human neuronal, platelet, placental, and pulmonary membranes (Ramamoorthy et al., 1993).

The SERT has been associated with depression and anxiety (Soubrie, 1988; Barnes, 1988); obesity (Blundell, 1986; Silverstone et al., 1986); alcoholism (Naranjo et al., 1987); postanoxic intention myoclonus (Van Woert et al., 1976); acute and chronic pain (Le Bars, 1988); as well as sleep disorders (Koella, 1988). SERT has also been shown to mediate behavioral and/or toxic effects of cocaine and amphetamines (Ramamoorthy et al., 1993). A variety of specific serotonin reuptake inhibitors (SSRIs) such as fluoxetine and paroxetine have been developed for the treatment of depression (reviewed in Schloss, 1998).

III. Nucleic Acid Molecules

The present invention provides nucleic acids encoding the serotonin transporter. Nucleic acids of the present invention may be derived from genomic DNA, complementary DNA (cDNA). More particularly, the present invention provides synthetic nucleic acid sequences comprising the amino acid sequences of the human and mouse serotonin transporter. An exemplary “wild-type” sequence is provided in SEQ ID NO:2, for example, that encoded by SEQ ID NO:1.

A “nucleic acid” as used herein includes single-stranded and double-stranded molecules, as well as DNA, RNA, chemically modified nucleic acids and nucleic acid analogs. It is contemplated that a nucleic acid within the scope of the present invention may be of about 20, of about 50 to about 90, of about 100 to about 200, of about 210 to about 300, of about 310 to about 350, of about 360, to about 400, of about 410 to about 450, of about 460 to about 500, of about 510 to about 550, of about 560 to about 600, of about 610 to about 650, of about 660 to about 700, of about 710 to about 750, of about 760 to about 800, of about 810 to about 850, of about 860 to about 900, about 1000, about 1100, about 1200, about 1300, about 1400, about 1500, about 1600, about 1700, about 1800, about 1900, about 2000 or greater nucleotide residues in length. Those of skill will recognize that in cases where the nucleic acid region encodes a serotonin transporter peptide, polypeptide or protein, the nucleic acid region can be quite long, depending upon the number of amino acids in the serotonin transporter molecule.

Similarly, a DNA segment comprising an isolated or purified serotonin transporter gene refers to a DNA segment including serotonin transporter protein, polypeptide or peptide coding sequences and, in certain aspects, regulatory sequences, isolated substantially away from other naturally occurring genes or protein encoding sequences. In this respect, the term “gene” is used for simplicity to refer to a functional protein, polypeptide or peptide encoding unit, as well as non-coding regulatory sequences. As will be understood by those in the art, this functional term includes both genomic sequences, cDNA sequences and engineered segments that express, or may be adapted to express, proteins, polypeptides, domains, peptides, fusion proteins and mutants of serotonin transporter encoded sequences.

“Isolated substantially away from other coding sequences” means that the gene of interest, in this case the serotonin transporter gene, forms the significant part of the coding region of the DNA segment, and that the DNA segment does not contain large portions of naturally-occurring coding DNA, such as large chromosomal fragments or other functional genes or cDNA coding regions. Of course, this refers to the DNA segment as originally isolated, and does not exclude genes or coding regions later added to the segment by the hand of man.

In particular embodiments, the invention concerns isolated DNA segments that encode a serotonin transporter protein, polypeptide or peptide that includes within its amino acid sequence a contiguous amino acid sequence in accordance with, or essentially as set forth in, SEQ ID NO: 2. Also included are non-coding segments such as introns and 5′ and 3′ regulatory sequences. In certain other embodiments, the invention concerns isolated DNA segments that include within their sequence a change relative to SEQ ID NO:2, selected from Gly56Ala (462G→C), Ile425Leu (1568A462G→C), Phe465Leu (1688T462G→C) and Leu550Val 1943G462G→C), two or more changes selected from Gly56Ala, Ile425Leu, Phe465Leu and Leu550Val, three or more changes selected from Gly56Ala, Ile425Leu, Phe465Leu and Leu550Val, or all of these changes. Other changes include nucleotide alterations selected from the following: 147C→A, 2517A→G, 15622G→A, 14519A→T, 14289A→C, 13912T→C, 13754C→T, IVS1a+20C→T, IVS1a+133G→A, IVS1a-47G→C, IVS1a-25G→A, IVS6-44G→C, ISV7+83C→T and ISV8-33C→T.

If desired, one also may prepare fusion proteins and peptides, e.g., where the nucleic acid encoding a serotonin transporter are aligned within the same expression unit with other proteins or peptides having desired functions, such as for purification or immunodetection purposes (e.g., proteins that may be purified by affinity chromatography and enzyme label coding regions, respectively).

In addition to nucleic acids encoding the serotonin transporter the present invention encompasses probes and primer that are complementary nucleic acids that stringent conditions to SERT sequences. As used herein, the term “complementary sequences” means nucleic acid sequences that are substantially complementary, as may be assessed by the same nucleotide comparison set forth above, or as defined as being capable of hybridizing to the contemplated nucleic acid segment of SEQ ID NO:1 under relatively stringent to stringent conditions such as those described herein.

The hybridizing segments often are shorter nucleic acids, or oligonucleotides. Sequences of 17 bases long should occur only once in the human genome and, therefore, suffice to specify a unique target sequence. However, it is contemplated that exemplary oligonucleotides of 8, 9, 10, 11, 12, 13, 14, 15, 16, 17, 18, 19, 20, 25, 30, 35, 40, 45, 50, 55, 60, 65, 70, 75, 80, 85, 90, 95, 100 or more base pairs can be used in accordance with the present invention.

Medium stringency conditions could be provided by about 0.1 to 0.25 M NaCl at temperatures of about 37° C. to about 55° C., while a low stringency condition could be provided by about 0.15 M to about 0.9 M salt, at temperatures ranging from about 20° C. to about 55° C. High stringency conditions typically are used for nucleic acid hybridization and are well known in the art. For example, conditions may comprise low salt and/or high temperature conditions, such as provided by about 0.02 M to about 0.15 M NaCl at temperatures of about 50° C. to about 70° C. It is understood that the temperature and ionic strength of a desired stringency are determined in part by the length of the particular nucleic acid(s), the length and nucleotide content of the target sequence(s), the charge composition of the nucleic acid(s), and to the presence or concentration of formamide, tetramethylammonium chloride or other solvent(s) in a hybridization mixture.

IV. Assessing Nucleic Acid Sequences

A. Methods of Assaying for SNPs

There are a large variety of techniques that can be used to assess genetic polymorphisms, and more are being discovered each day. The following is a very general discussion of a few of these techniques that can be used in accordance with the present invention.

1. RFLP

Restriction Fragment Length Polymorphism (RFLP) is a technique in which different DNA sequences may be differentiated by analysis of patterns derived from cleavage of that DNA. If two sequences differ in the distance between sites of cleavage of a particular restriction endonuclease, the length of the fragments produced will differ when the DNA is digested with a restriction enzyme. The similarity of the patterns generated can be used to differentiate species (and even strains) from one another.

Restriction endonucleases in turn are the enzymes that cleave DNA molecules at specific nucleotide sequences depending on the particular enzyme used. Enzyme recognition sites are usually 4 to 6 base pairs in length. Generally, the shorter the recognition sequence, the greater the number of fragments generated. If molecules differ in nucleotide sequence, fragments of different sizes may be generated. The fragments can be separated by gel electrophoresis. Restriction enzymes are isolated from a wide variety of bacterial genera and are thought to be part of the cell's defenses against invading bacterial viruses. Use of RFLP and restriction endonucleases in SNP analysis requires that the SNP affect cleavage of at least one restriction enzyme site.

2. Primer Extension

The primer and no more than three NTPs may be combined with a polymerase and the target sequence, which serves as a template for amplification. By using less than all four NTPs, it is possible to omit one or more of the polymorphic nucleotides needed for incorporation at the polymorphic site. It is important for the practice of the present invention that the amplification be designed such that the omitted nucleotide(s) is (are) not required between the 3′ end of the primer and the target polymorphism. The primer is then extended by a nucleic acid polymerase, in a preferred embodiment by Taq polymerase. If the omitted NTP is required at the polymorphic site, the primer is extended up to the polymorphic site, at which point the polymerization ceases. However, if the omitted NTP is not required at the polymorphic site, the primer will be extended beyond the polymorphic site, creating a longer product. Detection of the extension products is based on, for example, separation by size/length which will thereby reveal which polymorphism is present.

A specific form of primer extension, developed by the inventor, can be found in U.S. Ser. No. 10/407,846, which is hereby specifically incorporated by reference.

3. Oligonucleotide Hybridization

Oligonucleotides may be designed to hybridize directly to a target site of interest. The most common form of such analysis is where oligonucleotides are arrayed on a chip or plate in a “microarray.” Microarrays comprise a plurality of oligos spatially distributed over, and stably associated with, the surface of a substantially planar substrate, e.g., biochips. Microarrays of oligonucleotides have been developed and find use in a variety of applications, such as screening and DNA sequencing.

In gene analysis with microarrays, an array of “probe” oligonucleotides is contacted with a nucleic acid sample of interest, i.e., target. Contact is carried out under hybridization conditions and unbound nucleic acid is then removed. The resultant pattern of hybridized nucleic acid provides information regarding the genetic profile of the sample tested. Methodologies of gene analysis on microarrays are capable of providing both qualitative and quantitative information.

A variety of different arrays which may be used are known in the art. The probe molecules of the arrays which are capable of sequence specific hybridization with target nucleic acid may be polynucleotides or hybridizing analogues or mimetics thereof, including: nucleic acids in which the phosphodiester linkage has been replaced with a substitute linkage, such as phophorothioate, methylimino, methylphosphonate, phosphoramidate, guanidine and the like; nucleic acids in which the ribose subunit has been substituted, e.g., hexose phosphodiester; peptide nucleic acids; and the like. The length of the probes will generally range from 10 to 1000 nts, where in some embodiments the probes will be oligonucleotides and usually range from 15 to 150 nts and more usually from 15 to 100 nts in length, and in other embodiments the probes will be longer, usually ranging in length from 150 to 1000 nts, where the polynucleotide probes may be single- or double-stranded, usually single-stranded, and may be PCR fragments amplified from cDNA.

The probe molecules on the surface of the substrates will correspond to selected genes being analyzed and be positioned on the array at a known location so that positive hybridization events may be correlated to expression of a particular gene in the physiological source from which the target nucleic acid sample is derived. The substrates with which the probe molecules are stably associated may be fabricated from a variety of materials, including plastics, ceramics, metals, gels, membranes, glasses, and the like. The arrays may be produced according to any convenient methodology, such as preforming the probes and then stably associating them with the surface of the support or growing the probes directly on the support. A number of different array configurations and methods for their production are known to those of skill in the art and disclosed in U.S. Pat. Nos. 5,445,934, 5,532,128, 5,556,752, 5,242,974, 5,384,261, 5,405,783, 5,412,087, 5,424,186, 5,429,807, 5,436,327, 5,472,672, 5,527,681, 5,529,756, 5,545,531, 5,554,501, 5,561,071, 5,571,639, 5,593,839, 5,599,695, 5,624,711, 5,658,734, 5,700,637, and 6,004,755.

Following hybridization, where non-hybridized labeled nucleic acid is capable of emitting a signal during the detection step, a washing step is employed where unhybridized labeled nucleic acid is removed from the support surface, generating a pattern of hybridized nucleic acid on the substrate surface. A variety of wash solutions and protocols for their use are known to those of skill in the art and may be used.

Where the label on the target nucleic acid is not directly detectable, one then contacts the array, now comprising bound target, with the other member(s) of the signal producing system that is being employed. For example, where the label on the target is biotin, one then contacts the array with streptavidin-fluorescer conjugate under conditions sufficient for binding between the specific binding member pairs to occur. Following contact, any unbound members of the signal producing system will then be removed, e.g., by washing. The specific wash conditions employed will necessarily depend on the specific nature of the signal producing system that is employed, and will be known to those of skill in the art familiar with the particular signal producing system employed.

The resultant hybridization pattern(s) of labeled nucleic acids may be visualized or detected in a variety of ways, with the particular manner of detection being chosen based on the particular label of the nucleic acid, where representative detection means include scintillation counting, autoradiography, fluorescence measurement, calorimetric measurement, light emission measurement and the like.

Prior to detection or visualization, where one desires to reduce the potential for a mismatch hybridization event to generate a false positive signal on the pattern, the array of hybridized target/probe complexes may be treated with an endonuclease under conditions sufficient such that the endonuclease degrades single stranded, but not double stranded DNA. A variety of different endonucleases are known and may be used, where such nucleases include: mung bean nuclease, S1 nuclease, and the like. Where such treatment is employed in an assay in which the target nucleic acids are not labeled with a directly detectable label, e.g., in an assay with biotinylated target nucleic acids, the endonuclease treatment will generally be performed prior to contact of the array with the other member(s) of the signal producing system, e.g., fluorescent-streptavidin conjugate. Endonuclease treatment, as described above, ensures that only end-labeled target/probe complexes having a substantially complete hybridization at the 3′ end of the probe are detected in the hybridization pattern.

Following hybridization and any washing step(s) and/or subsequent treatments, as described above, the resultant hybridization pattern is detected. In detecting or visualizing the hybridization pattern, the intensity or signal value of the label will be not only be detected but quantified, by which is meant that the signal from each spot of the hybridization will be measured and compared to a unit value corresponding the signal emitted by known number of end-labeled target nucleic acids to obtain a count or absolute value of the copy number of each end-labeled target that is hybridized to a particular spot on the array in the hybridization pattern.

4. Sequencing

DNA sequencing enables one to perform a thorough analysis of DNA because it provides the most basic information of all: the sequence of nucleotides. Maxam & Gilbert developed the first widely used sequencing methods—a “chemical cleavage protocol.” Shortly thereafter, Sanger designed a procedure similar to the natural process of DNA replication. Even though both teams shared the 1980 Nobel Prize, Sanger's method became the standard because of its practicality.

Sanger's method, which is also referred to as dideoxy sequencing or chain termination, is based on the use of dideoxynucleotides (ddNTP's) in addition to the normal nucleotides (NTP's) found in DNA. Dideoxynucleotides are essentially the same as nucleotides except they contain a hydrogen group on the 3′ carbon instead of a hydroxyl group (OH). These modified nucleotides, when integrated into a sequence, prevent the addition of further nucleotides. This occurs because a phosphodiester bond cannot form between the dideoxynucleotide and the next incoming nucleotide, and thus the DNA chain is terminated. Using this method, optionally coupled with amplification of the nucleic acid target, one can now rapidly sequence large numbers of target molecules, usually employing automated sequencing apparati. Such techniques are well known to those of skill in the art.

B. Detection Systems

1. Mass Spectromety

By exploiting the intrinsic properties of mass and charge, mass spectrometry (MS) can resolved and confidently identified a wide variety of complex compounds, including nucleic acids and proteins. Traditional quantitative MS has used electrospray ionization (ESI) followed by tandem MS (MS/MS) (Chen et al., 2001; Zhong et al., 2001; Wu et al., 2000) while newer quantitative methods are being developed using matrix assisted laser desorption/ionization (MALDI) followed by time of flight (TOF) MS (Bucknall et al., 2002; Mirgorodskaya et al., 2000; Gobom et al., 2000).

i. ESI

ESI is a convenient ionization technique developed by Fenn and colleagues (Fenn et al., 1989) that is used to produce gaseous ions from highly polar, mostly nonvolatile biomolecules, including lipids. The sample is injected as a liquid at low flow rates (1-10 μL/min) through a capillary tube to which a strong electric field is applied. The field generates additional charges to the liquid at the end of the capillary and produces a fine spray of highly charged droplets that are electrostatically attracted to the mass spectrometer inlet. The evaporation of the solvent from the surface of a droplet as it travels through the desolvation chamber increases its charge density substantially. When this increase exceeds the Rayleigh stability limit, ions are ejected and ready for MS analysis.

A typical conventional ESI source consists of a metal capillary of typically 0.1-0.3 mm in diameter, with a tip held approximately 0.5 to 5 cm (but more usually 1 to 3 cm) away from an electrically grounded circular interface having at its center the sampling orifice, such as described by Kabarle et al. (1993). A potential difference of between 1 to 5 kV (but more typically 2 to 3 kV) is applied to the capillary by power supply to generate a high electrostatic field (10⁶ to 10⁷ V/m) at the capillary tip. A sample liquid carrying the analyte to be analyzed by the mass spectrometer, is delivered to tip through an internal passage from a suitable source (such as from a chromatograph or directly from a sample solution via a liquid flow controller). By applying pressure to the sample in the capillary, the liquid leaves the capillary tip as a small highly electrically charged droplets and further undergoes desolvation and breakdown to form single or multi-charged gas phase ions in the form of an ion beam. The ions are then collected by the grounded (or negatively-charged) interface plate and led through an the orifice into an analyzer of the mass spectrometer. During this operation, the voltage applied to the capillary is held constant. Aspects of construction of ESI sources are described, for example, in U.S. Pat. Nos. 5,838,002; 5,788,166; 5,757,994; RE 35,413; and 5,986,258.

ii. ESI/MS/MS

In ESI tandem mass spectroscopy (ESI/MS/MS), one is able to simultaneously analyze both precursor ions and product ions, thereby monitoring a single precursor product reaction and producing (through selective reaction monitoring (SRM)) a signal only when the desired precursor ion is present. When the internal standard is a stable isotope-labeled version of the analyte, this is known as quantification by the stable isotope dilution method. This approach has been used to accurately measure pharmaceuticals (Zweigenbaum et al., 2000; Zweigenbaum et al., 1999) and bioactive peptides (Desiderio et al., 1996; Lovelace et al., 1991). Newer methods are performed on widely available MALDI-TOF instruments, which can resolve a wider mass range and have been used to quantify metabolites, peptides, and proteins. Larger molecules such as peptides can be quantified using unlabeled homologous peptides as long as their chemistry is similar to the analyte peptide (Duncan et al., 1993; Bucknall et al., 2002). Protein quantification has been achieved by quantifying tryptic peptides (Mirgorodskaya et al., 2000). Complex mixtures such as crude extracts can be analyzed, but in some instances sample clean up is required (Nelson et al., 1994; Gobom et al., 2000).

iii. SIMS

Secondary ion mass spectroscopy, or SIMS, is an analytical method that uses ionized particles emitted from a surface for mass spectroscopy at a sensitivity of detection of a few parts per billion. The sample surface is bombarded by primary energetic particles, such as electrons, ions (e.g., O, Cs), neutrals or even photons, forcing atomic and molecular particles to be ejected from the surface, a process called sputtering. Since some of these sputtered particles carry a charge, a mass spectrometer can be used to measure their mass and charge. Continued sputtering permits measuring of the exposed elements as material is removed. This in turn permits one to construct elemental depth profiles. Although the majority of secondary ionized particles are electrons, it is the secondary ions which are detected and analysis by the mass spectrometer in this method.

iv. LD-MS and LDLPMS

Laser desorption mass spectroscopy (LD-MS) involves the use of a pulsed laser, which induces desorption of sample material from a sample site—effectively, this means vaporization of sample off of the sample substrate. This method is usually only used in conjunction with a mass spectrometer, and can be performed simultaneously with ionization if one uses the right laser radiation wavelength.

When coupled with Time-of-Flight (TOF) measurement, LD-MS is referred to as LDLPMS (Laser Desorption Laser Photoionization Mass Spectroscopy). The LDLPMS method of analysis gives instantaneous volatilization of the sample, and this form of sample fragmentation permits rapid analysis without any wet extraction chemistry. The LDLPMS instrumentation provides a profile of the species present while the retention time is low and the sample size is small. In LDLPMS, an impactor strip is loaded into a vacuum chamber. The pulsed laser is fired upon a certain spot of the sample site, and species present are desorbed and ionized by the laser radiation. This ionization also causes the molecules to break up into smaller fragment-ions. The positive or negative ions made are then accelerated into the flight tube, being detected at the end by a microchannel plate detector. Signal intensity, or peak height, is measured as a function of travel time. The applied voltage and charge of the particular ion determines the kinetic energy, and separation of fragments are due to different size causing different velocity. Each ion mass will thus have a different flight-time to the detector.

One can either form positive ions or negative ions for analysis. Positive ions are made from regular direct photoionization, but negative ion formation require a higher powered laser and a secondary process to gain electrons. Most of the molecules that come off the sample site are neutrals, and thus can attract electrons based on their electron affinity. The negative ion formation process is less efficient than forming just positive ions. The sample constituents will also affect the outlook of a negative ion spectra.

Other advantages with the LDLPMS method include the possibility of constructing the system to give a quiet baseline of the spectra because one can prevent coevolved neutrals from entering the flight tube by operating the instrument in a linear mode. Also, in environmental analysis, the salts in the air and as deposits will not interfere with the laser desorption and ionization. This instrumentation also is very sensitive, known to detect trace levels in natural samples without any prior extraction preparations.

v. MALDI-TOF-MS

Since its inception and commercial availability, the versatility of MALDI-TOF-MS has been demonstrated convincingly by its extensive use for qualitative analysis. For example, MALDI-TOF-MS has been employed for the characterization of synthetic polymers (Marie et al., 2000; Wu et al., 1998). peptide and protein analysis (Roepstorff et al., 2000; Nguyen et al., 1995), DNA and oligonucleotide sequencing (Miketova et al., 1997; Faulstich et al., 1997; Bentzley et al., 1996), and the characterization of recombinant proteins (Kanazawa et al., 1999; Villanueva et al., 1999). Recently, applications of MALDI-TOF-MS have been extended to include the direct analysis of biological tissues and single cell organisms with the aim of characterizing endogenous peptide and protein constituents (Li et al., 2000; Lynn et al., 1999; Stoeckli et al., 2001; Caprioli et al., 1997; Chaurand et al., 1999; Jespersen et al., 1999).

The properties that make MALDI-TOF-MS a popular qualitative tool—its ability to analyze molecules across an extensive mass range, high sensitivity, minimal sample preparation and rapid analysis times—also make it a potentially useful quantitative tool. MALDI-TOF-MS also enables non-volatile and thermally labile molecules to be analyzed with relative ease. It is therefore prudent to explore the potential of MALDI-TOF-MS for quantitative analysis in clinical settings, for toxicological screenings, as well as for environmental analysis. In addition, the application of MALDI-TOF-MS to the quantification of peptides and proteins is particularly relevant. The ability to quantify intact proteins in biological tissue and fluids presents a particular challenge in the expanding area of proteomics and investigators urgently require methods to accurately measure the absolute quantity of proteins. While there have been reports of quantitative MALDI-TOF-MS applications, there are many problems inherent to the MALDI ionization process that have restricted its widespread use (Kazmaier et al., 1998; Horak et al., 2001; Gobom et al., 2000; Wang et al., 2000; Desiderio et al., 2000). These limitations primarily stem from factors such as the sample/matrix heterogeneity, which are believed to contribute to the large variability in observed signal intensities for analytes, the limited dynamic range due to detector saturation, and difficulties associated with coupling MALDI-TOF-MS to on-line separation techniques such as liquid chromatography. Combined, these factors are thought to compromise the accuracy, precision, and utility with which quantitative determinations can be made.

Because of these difficulties, practical examples of quantitative applications of MALDI-TOF-MS have been limited. Most of the studies to date have focused on the quantification of low mass analytes, in particular, alkaloids or active ingredients in agricultural or food products (Wang et al., 1999; Jiang et al., 2000; Wang et al., 2000; Yang et al., 2000; Wittmann et al., 2001), whereas other studies have demonstrated the potential of MALDI-TOF-MS for the quantification of biologically relevant analytes such as neuropeptides, proteins, antibiotics, or various metabolites in biological tissue or fluid (Muddiman et al., 1996; Nelson et al., 1994; Duncan et al., 1993; Gobom et al., 2000; Wu et al., 1997; Mirgorodskaya et al., 2000). In earlier work it was shown that linear calibration curves could be generated by MALDI-TOF-MS provided that an appropriate internal standard was employed (Duncan et al., 1993). This standard can “correct” for both sample-to-sample and shot-to-shot variability. Stable isotope labeled internal standards (isotopomers) give the best result.

With the marked improvement in resolution available on modern commercial instruments, primarily because of delayed extraction (Bahr et al., 1997; Takach et al., 1997), the opportunity to extend quantitative work to other examples is now possible; not only of low mass analytes, but also biopolymers. Of particular interest is the prospect of absolute multi-component quantification in biological samples (e.g., proteomics applications).

The properties of the matrix material used in the MALDI method are critical. Only a select group of compounds is useful for the selective desorption of proteins and polypeptides. A review of all the matrix materials available for peptides and proteins shows that there are certain characteristics the compounds must share to be analytically useful. Despite its importance, very little is known about what makes a matrix material “successful” for MALDI. The few materials that do work well are used heavily by all MALDI practitioners and new molecules are constantly being evaluated as potential matrix candidates. With a few exceptions, most of the matrix materials used are solid organic acids. Liquid matrices have also been investigated, but are not used routinely.

2. Hybridization

There are a variety of ways by which one can assess genetic profiles, and may of these rely on nucleic acid hybridization. Hybridization is defined as the ability of a nucleic acid to selectively form duplex molecules with complementary stretches of DNAs and/or RNAs. Depending on the application envisioned, one would employ varying conditions of hybridization to achieve varying degrees of selectivity of the probe or primers for the target sequence.

Typically, a probe or primer of between 13 and 100 nucleotides, preferably between 17 and 100 nucleotides in length up to 1-2 kilobases or more in length will allow the formation of a duplex molecule that is both stable and selective. Molecules having complementary sequences over contiguous stretches greater than 20 bases in length are generally preferred, to increase stability and selectivity of the hybrid molecules obtained. One will generally prefer to design nucleic acid molecules for hybridization having one or more complementary sequences of 20 to 30 nucleotides, or even longer where desired. Such fragments may be readily prepared, for example, by directly synthesizing the fragment by chemical means or by introducing selected sequences into recombinant vectors for recombinant production.

For applications requiring high selectivity, one will typically desire to employ relatively high stringency conditions to form the hybrids. For example, relatively low salt and/or high temperature conditions, such as provided by about 0.02 M to about 0.10 M NaCl at temperatures of about 50° C. to about 70° C. Such high stringency conditions tolerate little, if any, mismatch between the probe or primers and the template or target strand and would be particularly suitable for isolating specific genes or for detecting specific mRNA transcripts. It is generally appreciated that conditions can be rendered more stringent by the addition of increasing amounts of formamide.

For certain applications, for example, lower stringency conditions may be used. Under these conditions, hybridization may occur even though the sequences of the hybridizing strands are not perfectly complementary, but are mismatched at one or more positions. Conditions may be rendered less stringent by increasing salt concentration and/or decreasing temperature. For example, a medium stringency condition could be provided by about 0.1 to 0.25 M NaCl at temperatures of about 37° C. to about 55° C., while a low stringency condition could be provided by about 0.15 M to about 0.9 M salt, at temperatures ranging from about 20° C. to about 55° C. Hybridization conditions can be readily manipulated depending on the desired results.

In other embodiments, hybridization may be achieved under conditions of, for example, 50 mM Tris-HCl (pH 8.3), 75 mM KCl, 3 mM MgCl₂, 1.0 mM dithiothreitol, at temperatures between approximately 20° C. to about 37° C. Other hybridization conditions utilized could include approximately 10 mM Tris-HCl (pH 8.3), 50 mM KCl, 1.5 mM MgCl₂, at temperatures ranging from approximately 40° C. to about 72° C.

In certain embodiments, it will be advantageous to employ nucleic acids of defined sequences of the present invention in combination with an appropriate means, such as a label, for determining hybridization. A wide variety of appropriate indicator means are known in the art, including fluorescent, radioactive, enzymatic or other ligands, such as avidin/biotin, which are capable of being detected. In preferred embodiments, one may desire to employ a fluorescent label or an enzyme tag such as urease, alkaline phosphatase or peroxidase, instead of radioactive or other environmentally undesirable reagents. In the case of enzyme tags, colorimetric indicator substrates are known that can be employed to provide a detection means that is visibly or spectrophotometrically detectable, to identify specific hybridization with complementary nucleic acid containing samples.

In general, it is envisioned that the probes or primers described herein will be useful as reagents in solution hybridization, as in PCRT™, for detection of expression of corresponding genes, as well as in embodiments employing a solid phase. In embodiments involving a solid phase, the test DNA (or RNA) is adsorbed or otherwise affixed to a selected matrix or surface. This fixed, single-stranded nucleic acid is then subjected to hybridization with selected probes under desired conditions. The conditions selected will depend on the particular circumstances (depending, for example, on the G+C content, type of target nucleic acid, source of nucleic acid, size of hybridization probe, etc.). Optimization of hybridization conditions for the particular application of interest is well known to those of skill in the art. After washing of the hybridized molecules to remove non-specifically bound probe molecules, hybridization is detected, and/or quantified, by determining the amount of bound label. Representative solid phase hybridization methods are disclosed in U.S. Pat. Nos. 5,843,663, 5,900,481 and 5,919,626. Other methods of hybridization that may be used in the practice of the present invention are disclosed in U.S. Pat. Nos. 5,849,481, 5,849,486 and 5,851,772. The relevant portions of these and other references identified in this section of the Specification are incorporated herein by reference.

3. Detectable Labels

Various nucleic acids may be visualized in order to confirm their presence, quantity or sequence. In one embodiment, the primer is conjugated to a chromophore but may instead be radiolabeled or fluorometrically labeled. In another embodiment, the primer is conjugated to a binding partner that carries a detectable moiety, such as an antibody or biotin. In other embodiments, the primer incorporates a fluorescent dye or label. In yet other embodiments, the primer has a mass label that can be used to detect the molecule amplified. Other embodiments also contemplate the use of Taqman™ and Molecular Beacon™ probes. Alternatively, one or more of the dNTPs may be labeled with a radioisotope, a fluorophore, a chromophore, a dye or an enzyme. Also, chemicals whose properties change in the presence of DNA can be used for detection purposes. For example, the methods may involve staining of a gel with, or incorporation into the separation media, a fluorescent dye, such as ethidium bromide or Vistra Green, and visualization under an appropriate light source.

The choice of label incorporated into the products is dictated by the method used for analysis. When using capillary electrophoresis, microfluidic electrophoresis, HPLC, or LC separations, either incorporated or intercalated fluorescent dyes are used to label and detect the amplification products. Samples are detected dynamically, in that fluorescence is quantitated as a labeled species moves past the detector. If any electrophoretic method, HPLC, or LC is used for separation, products can be detected by absorption of UV light, a property inherent to DNA and therefore not requiring addition of a label. If polyacrylamide gel or slab gel electrophoresis is used, the primer for the extension reaction can be labeled with a fluorophore, a chromophore or a radioisotope, or by associated enzymatic reaction. Alternatively, if polyacrylamide gel or slab gel electrophoresis is used, one or more of the NTPs in the extension reaction can be labeled with a fluorophore, a chromophore or a radioisotope, or by associated enzymatic reaction. Enzymatic detection involves binding an enzyme to a nucleic acid, e.g., via a biotin:avidin interaction, following separation of the amplification products on a gel, then detection by chemical reaction, such as chemiluminescence generated with luminol. A fluorescent signal can be monitored dynamically. Detection with a radioisotope or enzymatic reaction requires an initial separation by gel electrophoresis, followed by transfer of DNA molecules to a solid support (blot) prior to analysis. If blots are made, they can be analyzed more than once by probing, stripping the blot, and then reprobing. If the extension products are separated using a mass spectrometer no label is required because nucleic acids are detected directly.

In the case of radioactive isotopes, tritium, ¹⁴C and ³²P are used predominantly. Among the fluorescent labels contemplated for use as conjugates include Alexa 350, Alexa 430, AMCA, BODIPY 630/650, BODIPY 650/665, BODIPY-FL, BODIPY—R6G, BODIPY-TMR, BODIPY-TRX, Cascade Blue, Cy3, Cy5,6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, TAMRA, TET, Tetramethylrhodamine, and/or Texas Red.

4. Other Methods of Detecting Nucleic Acids

Other methods of nucleic acid detection that may be used in the practice of the instant invention are disclosed in U.S. Pat. Nos. 5,840,873, 5,843,640, 5,843,651, 5,846,708, 5,846,717, 5,846,726, 5,846,729, 5,849,487, 5,853,990, 5,853,992, 5,853,993, 5,856,092, 5,861,244, 5,863,732, 5,863,753, 5,866,331, 5,905,024, 5,910,407, 5,912,124, 5,912,145, 5,919,630, 5,925,517, 5,928,862, 5,928,869, 5,929,227, 5,932,413 and 5,935,791, each of which is incorporated herein by reference in its entirety.

5. Oligonucleotide Synthesis

Oligonucleotide synthesis is well known to those of skill in the art. Various mechanisms of oligonucleotide synthesis have been disclosed in for example, U.S. Pat. Nos. 4,659,774, 4,816,571, 5,141,813, 5,264,566, 4,959,463, 5,428,148, 5,554,744, 5,574,146, 5,602,244, each of which is incorporated herein by reference in its entirety. Basically, chemical synthesis can be achieved by the diester method, the triester method polynucleotides phosphorylase method and by solid-phase chemistry. These methods are discussed in further detail below.

Diester method. The diester method was the first to be developed to a usable state, primarily by Khorana and co-workers (Khorana, 1979). The basic step is the joining of two suitably protected deoxynucleotides to form a dideoxynucleotide containing a phosphodiester bond. The diester method is well established and has been used to synthesize DNA molecules (Khorana, 1979).

Triester method. The main difference between the diester and triester methods is the presence in the latter of an extra protecting group on the phosphate atoms of the reactants and products (Itakura et al., 1975). The phosphate protecting group is usually a chlorophenyl group, which renders the nucleotides and polynucleotide intermediates soluble in organic solvents. Therefore, purifications are done in chloroform solutions. Other improvements in the method include (i) the block coupling of trimers and larger oligomers, (ii) the extensive use of high-performance liquid chromatography for the purification of both intermediate and final products, and (iii) solid-phase synthesis.

Polynucleotide phosphorylase method. This is an enzymatic method of DNA synthesis that can be used to synthesize many useful oligodeoxynucleotides (Gillam et al., 1978). Under controlled conditions, polynucleotide phosphorylase adds predominantly a single nucleotide to a short oligodeoxynucleotide. Chromatographic purification allows the desired single adduct to be obtained. At least a trimer is required to initiate the method of adding one base at a time, a primer that must be obtained by some other method. The polynucleotide phosphorylase method works and has the advantage that the procedures involved are familiar to most biochemists.

Solid-phase methods. The technology developed for the solid-phase synthesis of polypeptides has been applied after an, it has been possible to attach the initial nucleotide to solid support material has been attached by proceeding with the stepwise addition of nucleotides. All mixing and washing steps are simplified, and the procedure becomes amenable to automation. These syntheses are now routinely carried out using automatic DNA synthesizers.

Phosphoramidite chemistry (Beaucage, 1993) has become by far the most widely used coupling chemistry for the synthesis of oligonucleotides. As is well known to those skilled in the art, phosphoramidite synthesis of oligonucleotides involves activation of nucleoside phosphoramidite monomer precursors by reaction with an activating agent to form activated intermediates, followed by sequential addition of the activated intermediates to the growing oligonucleotide chain (generally anchored at one end to a suitable solid support) to form the oligonucleotide product.

6. Separation of Nucleic Acids

In certain embodiments, nucleic acid products are separated by agarose, agarose-acrylamide or polyacrylamide gel electrophoresis using standard methods (Sambrook et al., 1989). Separated products may be cut out and eluted from the gel for further manipulation. Using low melting point agarose gels, the skilled artisan my remove the separated band by heating the gel, followed by extraction of the nucleic acid.

Separation of nucleic acids may also be effected by chromatographic techniques known in the art. There are many kinds of chromatography that may be used in the practice of the present invention, including capillary adsorption, partition, ion-exchange, hydroxylapatite, molecular sieve, reverse-phase, column, paper, thin-layer, and gas chromatography as well as HPLC.

A number of the above separation platforms can be coupled to achieve separations based on two different properties. For example, some of the primers can be coupled with a moiety that allows affinity capture, and some primers remain unmodified. Modifications can include a sugar (for binding to a lectin column), a hydrophobic group (for binding to a reverse-phase column), biotin (for binding to a streptavidin column), or an antigen (for binding to an antibody column). Samples are run through an affinity chromatography column. The flow-through fraction is collected, and the bound fraction eluted (by chemical cleavage, salt elution, etc.). Each sample is then further fractionated based on a property, such as mass, to identify individual components.

V. Amplifying a Target Sequence

In a particular embodiment, it may be desirable to amplify the target sequence before evaluating the SNP. Nucleic acids used as a template for amplification may be isolated from cells, tissues or other samples according to standard methodologies (Sambrook et al., 1989). In certain embodiments, analysis is performed on whole cell or tissue homogenates or biological fluid samples without substantial purification of the template nucleic acid. The nucleic acid may be genomic DNA or fractionated or whole cell RNA. Where RNA is used, it may be desired to first convert the RNA to a complementary DNA. The DNA also may be from a cloned source or synthesized in vitro.

The term “primer,” as used herein, is meant to encompass any nucleic acid that is capable of priming the synthesis of a nascent nucleic acid in a template-dependent process. Typically, primers are oligonucleotides from ten to twenty or thirty base pairs in length, but longer sequences can be employed. Primers may be provided in double-stranded or single-stranded form, although the single-stranded form is preferred.

Pairs of primers designed to selectively hybridize to nucleic acids flanking the polymorphic site are contacted with the template nucleic acid under conditions that permit selective hybridization. Depending upon the desired application, high stringency hybridization conditions may be selected that will only allow hybridization to sequences that are completely complementary to the primers. In other embodiments, hybridization may occur under reduced stringency to allow for amplification of nucleic acids containing one or more mismatches with the primer sequences. Once hybridized, the template-primer complex is contacted with one or more enzymes that facilitate template-dependent nucleic acid synthesis. Multiple rounds of amplification, also referred to as “cycles,” are conducted until a sufficient amount of amplification product is produced.

It is also possible that multiple target sequences will be amplified in a single reaction. Primers designed to expand specific sequences located in different regions of the target genome, thereby identifying different polymorphisms, would be mixed together in a single reaction mixture. The resulting amplification mixture would contain multiple amplified regions, and could be used as the source template for polymorphism detection using the methods described in this application.

A number of template dependent processes are available to amplify the oligonucleotide sequences present in a given template sample. One of the best known amplification methods is the polymerase chain reaction (referred to as PCR™), which is described in detail in U.S. Pat. Nos. 4,683,195, 4,683,202 and 4,800,159, and in Innis et al., 1988, each of which is incorporated herein by reference in their entirety.

A reverse transcriptase PCR™ amplification procedure may be performed when the source of nucleic acid is fractionated or whole cell RNA. Methods of reverse transcribing RNA into cDNA are well known (see Sambrook et al., 1989). Alternative methods for reverse polymerization utilize thermostable DNA polymerases. These methods are described in WO 90/07641. Polymerase chain reaction methodologies are well known in the art. Representative methods of RT-PCR are described in U.S. Pat. No. 5,882,864.

Another method for amplification is ligase chain reaction (“LCR”), disclosed in European Application No. 320 308, incorporated herein by reference in its entirety. U.S. Pat. No. 4,883,750 describes a method similar to LCR for binding probe pairs to a target sequence. A method based on PCR™ and oligonucleotide ligase assay (OLA), disclosed in U.S. Pat. No. 5,912,148, may also be used.

Another ligase-mediated reaction is disclosed by Guilfoyle et al. (1997). Genomic DNA is digested with a restriction enzyme and universal linkers are then ligated onto the restriction fragments. Primers to the universal linker sequence are then used in PCR to amplify the restriction fragments. By varying the conditions of the PCR, one can specifically amplify fragments of a certain size (i.e., less than a 1000 bases). An example for use with the present invention would be to digest genomic DNA with XbaI, and ligate on M13-universal primers with an XbaI over hang, followed by amplification of the genomic DNA with an M13 universal primer. Only a small percentage of the total DNA would be amplified (the restriction fragments that were less than 1000 bases). One would then use labeled primers that correspond to a SNP are located within XbaI restriction fragments of a certain size (<1000 bases) to perform the assay. The benefit to using this approach is that each individual region would not have to be amplified separately. There would be the potential to screen thousands of SNPs from the single PCR reaction, i.e., multiplex potential.

Alternative methods for amplification of target nucleic acid sequences that may be used in the practice of the present invention are disclosed in U.S. Pat. Nos. 5,843,650, 5,846,709, 5,846,783, 5,849,546, 5,849,497, 5,849,547, 5,858,652, 5,866,366, 5,916,776, 5,922,574, 5,928,905, 5,928,906, 5,932,451, 5,935,825, 5,939,291 and 5,942,391, GB Application No. 2 202 328, and in PCT Application No. PCT/US89/01025, each of which is incorporated herein by reference in its entirety.

Qbeta Replicase, described in PCT Application No. PCT/US87/00880, may also be used as an amplification method in the present invention. In this method, a replicative sequence of RNA that has a region complementary to that of a target is added to a sample in the presence of an RNA polymerase. The polymerase will copy the replicative sequence, which may then be detected.

An isothermal amplification method, in which restriction endonucleases and ligases are used to achieve the amplification of target molecules that contain nucleotide 5′-[alpha-thio]-triphosphates in one strand of a restriction site may also be useful in the amplification of nucleic acids in the present invention (Walker et al., 1992). Strand Displacement Amplification (SDA), disclosed in U.S. Pat. No. 5,916,779, is another method of carrying out isothermal amplification of nucleic acids which involves multiple rounds of strand displacement and synthesis, i.e., nick translation.

Other nucleic acid amplification procedures include polymerization-based amplification systems (TAS), including nucleic acid sequence based amplification (NASBA) and 3SR (Kwoh et al., 1989; Gingeras et al., PCT Application WO 88/10315, incorporated herein by reference in their entirety). European Application No. 329 822 discloses a nucleic acid amplification process involving cyclically synthesizing single-stranded RNA (ssRNA), ssDNA, and double-stranded DNA (dsDNA), which may be used in accordance with the present invention.

PCT Application WO 89/06700 (incorporated herein by reference in its entirety) discloses a nucleic acid sequence amplification scheme based on the hybridization of a promoter region/primer sequence to a target single-stranded DNA (ssDNA) followed by polymerization of many RNA copies of the sequence. This scheme is not cyclic, i.e., new templates are not produced from the resultant RNA transcripts. Other amplification methods include “race” and “one-sided PCR” (Frohman, 1990; Ohara et al., 1989).

Another advantageous step is to prevent unincorporated NTPs from being incorporated in a subsequent primer extension reaction. Commercially available kits may be used to remove unincorporated NTPs from the amplification products. The use of shrimp alkaline phosphatase to destroy unincorporated NTPs is also a well-known strategy for this purpose.

VI. Polypeptides Encoding a Serotonin Transporter and Detection Thereof

The present invention provides amino acid sequences of the serotonin transporter. More particularly, the present invention provides amino acid sequences of the human serotonin transporter as in SEQ ID NO: 2, and changes including: Gly56Ala (462G→C), Ile425Leu (1568A462G→C), Phe465Leu (1688T462G→C) and Leu550Val 1943G462G→C), two or more changes selected from Gly56Ala, Ile425Leu, Phe465Leu and Leu550Val, three or more changes selected from Gly56Ala, Ile425Leu, Phe465Leu and Leu550Val, or all of these changes. Accordingly, the term “amino acid composition” encompasses amino molecule sequences comprising at least one of the 20 common amino acids in naturally synthesized proteins, or at least one modified or unusual amino acid. It is also well understood that where certain residues are shown to be particularly important to the biological or structural properties of a protein, polypeptide or peptide, e.g., residues in binding regions or active sites, such residues may not generally be exchanged. In this manner, functional equivalents are defined herein as those peptides which maintain a substantial amount of their native biological activity.

In certain embodiments the amino acid composition of the present invention comprises at least one protein, polypeptide or peptide. In further embodiments the amino acid composition comprises a biocompatible protein, polypeptide or peptide. As used herein, the term “biocompatible” refers to a substance which produces no significant untoward effects when applied to, or administered to, a given organism according to the methods and amounts described herein. Such untoward or undesirable effects are those such as significant toxicity or adverse immunological reactions. In preferred embodiments, biocompatible protein, polypeptide or peptide containing compositions will generally be mammalian proteins or peptides or synthetic proteins or peptides each essentially free from toxins, pathogens and harmful immunogens.

In certain embodiments, amino acid sequence variants of the protein, polypeptide, or peptide may be prepared. Such variants may can be substitutional, insertional or deletion variants are methods of preparing these variants are well known in the art. These variants include polymorphisms and mutants that affect the function and activity of the serotonin transporter. Deletion variants lack one or more residues of the native protein which are not essential for function or immunogenic activity. Insertional variants typically involve the addition of material at a non-terminal point in the polypeptide. Insertional variants include fusion proteins, or hybrid proteins containing sequences from other proteins and polypeptides which are homologues of the polypeptide.

Substitutional variants typically contain the exchange of one amino acid for another at one or more sites within the protein, and may be designed to modulate one or more properties of the polypeptide such as stability against proteolytic cleavage. Substitutions envisioned in the present invention include Gly56Ala (462G→C), Ile425Leu (1568A462G→C), Phe465Leu (1688T462G→C) and Leu550Val 1943G462G→C), two or more changes selected from Gly56Ala, Ile425Leu, Phe465Leu and Leu550Val, three or more changes selected from Gly56Ala, Ile425Leu, Phe465Leu and Leu550Val, or all of these changes.

There are a variety of methods that can be used to assess protein expression. One such approach is to perform protein identification with the use of antibodies. As used herein, the term “antibody” is intended to refer broadly to any immunologic binding agent such as IgG, IgM, IgA, IgD and IgE. Generally, IgG and/or IgM are preferred because they are the most common antibodies in the physiological situation and because they are most easily made in a laboratory setting. The term “antibody” also refers to any antibody-like molecule that has an antigen binding region, and includes antibody fragments such as Fab′, Fab, F(ab′)₂, single domain antibodies (DABs), Fv, scFv (single chain Fv), and the like. The techniques for preparing and using various antibody-based constructs and fragments are well known in the art. Means for preparing and characterizing antibodies, both polyclonal and monoclonal, are also well known in the art (see, e.g., Antibodies: A Laboratory Manual, Cold Spring Harbor Laboratory, 1988; incorporated herein by reference). In particular, antibodies to calcyclin, calpactin I light chain, astrocytic phosphoprotein PEA-15 and tubulin-specific chaperone A are contemplated.

In accordance with the present invention, immunodetection methods are provided. Some immunodetection methods include enzyme linked immunosorbent assay (ELISA), radioimmunoassay (RIA), immunoradiometric assay, fluoroimmunoassay, chemiluminescent assay, bioluminescent assay, and Western blot to mention a few. The steps of various useful immunodetection methods have been described in the scientific literature, such as, e.g., Doolittle & Ben-Zeev 0 (1999); Gulbis & Galand (1993); De Jager et al. (1993); and Nakamura et al. (1987), each incorporated herein by reference.

In general, the immunobinding methods include obtaining a sample suspected of containing a relevant polypeptide, and contacting the sample with a first antibody under conditions effective to allow the formation of immunocomplexes. In terms of antigen detection, the biological sample analyzed may be any sample that is suspected of containing an antigen, such as, for example, a tissue section or specimen, a homogenized tissue extract, a cell, or even a biological fluid.

Contacting the chosen biological sample with the antibody under effective conditions and for a period of time sufficient to allow the formation of immune complexes (primary immune complexes) is generally a matter of simply adding the antibody composition to the sample and incubating the mixture for a period of time long enough for the antibodies to form immune complexes with, i.e., to bind to, any antigens present. After this time, the sample-antibody composition, such as a tissue section, ELISA plate, dot blot or western blot, will generally be washed to remove any non-specifically bound antibody species, allowing only those antibodies specifically bound within the primary immune complexes to be detected.

In general, the detection of immunocomplex formation is well known in the art and may be achieved through the application of numerous approaches. These methods are generally based upon the detection of a label or marker, such as any of those radioactive, fluorescent, biological and enzymatic tags. U.S. Patents concerning the use of such labels include U.S. Pat. Nos. 3,817,837; 3,850,752; 3,939,350; 3,996,345; 4,277,437; 4,275,149 and 4,366,241, each incorporated herein by reference. Of course, one may find additional advantages through the use of a secondary binding ligand such as a second antibody and/or a biotin/avidin ligand binding arrangement, as is known in the art.

The antibody employed in the detection may itself be linked to a detectable label, wherein one would then simply detect this label, thereby allowing the amount of the primary immune complexes in the composition to be determined. Alternatively, the first antibody that becomes bound within the primary immune complexes may be detected by means of a second binding ligand that has binding affinity for the antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under effective conditions and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Further methods include the detection of primary immune complexes by a two step approach. A second binding ligand, such as an antibody, that has binding affinity for the antibody is used to form secondary immune complexes, as described above. After washing, the secondary immune complexes are contacted with a third binding ligand or antibody that has binding affinity for the second antibody, again under effective conditions and for a period of time sufficient to allow the formation of immune complexes (tertiary immune complexes). The third ligand or antibody is linked to a detectable label, allowing detection of the tertiary immune complexes thus formed. This system may provide for signal amplification if this is desired.

One method of immunodetection designed by Charles Cantor uses two different antibodies. A first step biotinylated, monoclonal or polyclonal antibody is used to detect the target antigen(s), and a second step antibody is then used to detect the biotin attached to the complexed biotin. In that method the sample to be tested is first incubated in a solution containing the first step antibody. If the target antigen is present, some of the antibody binds to the antigen to form a biotinylated antibody/antigen complex. The antibody/antigen complex is then amplified by incubation in successive solutions of streptavidin (or avidin), biotinylated DNA, and/or complementary biotinylated DNA, with each step adding additional biotin sites to the antibody/antigen complex. The amplification steps are repeated until a suitable level of amplification is achieved, at which point the sample is incubated in a solution containing the second step antibody against biotin. This second step antibody is labeled, as for example with an enzyme that can be used to detect the presence of the antibody/antigen complex by histoenzymology using a chromogen substrate. With suitable amplification, a conjugate can be produced which is macroscopically visible.

Another known method of immunodetection takes advantage of the immuno-PCR (Polymerase Chain Reaction) methodology. The PCR method is similar to the Cantor method up to the incubation with biotinylated DNA, however, instead of using multiple rounds of streptavidin and biotinylated DNA incubation, the DNA/biotin/streptavidin/antibody complex is washed out with a low pH or high salt buffer that releases the antibody. The resulting wash solution is then used to carry out a PCR reaction with suitable primers with appropriate controls. At least in theory, the enormous amplification capability and specificity of PCR can be utilized to detect a single antigen molecule.

As detailed above, immunoassays are in essence binding assays. Certain immunoassays are the various types of enzyme linked immunosorbent assays (ELISAs) and radioimmunoassays (RIA) known in the art. However, it will be readily appreciated that detection is not limited to such techniques, and Western blotting, dot blotting, FACS analyses, and the like may also be used.

In one exemplary ELISA, the antibodies of the invention are immobilized onto a selected surface exhibiting protein affinity, such as a well in a polystyrene microtiter plate. Then, a test composition suspected of containing the antigen, such as a clinical sample, is added to the wells. After binding and washing to remove non-specifically bound immune complexes, the bound antigen may be detected. Detection is generally achieved by the addition of another antibody that is linked to a detectable label. This type of ELISA is a simple “sandwich ELISA.” Detection may also be achieved by the addition of a second antibody, followed by the addition of a third antibody that has binding affinity for the second antibody, with the third antibody being linked to a detectable label.

In another exemplary ELISA, the samples suspected of containing the antigen are immobilized onto the well surface and then contacted with the anti-ORF message and anti-ORF translated product antibodies of the invention. After binding and washing to remove non-specifically bound immune complexes, the bound anti-ORF message and anti-ORF translated product antibodies are detected. Where the initial anti-ORF message and anti-ORF translated product antibodies are linked to a detectable label, the immune complexes may be detected directly. Again, the immune complexes may be detected using a second antibody that has binding affinity for the first anti-ORF message and anti-ORF translated product antibody, with the second antibody being linked to a detectable label.

Another ELISA in which the antigens are immobilized, involves the use of antibody competition in the detection. In this ELISA, labeled antibodies against an antigen are added to the wells, allowed to bind, and detected by means of their label. The amount of an antigen in an unknown sample is then determined by mixing the sample with the labeled antibodies against the antigen during incubation with coated wells. The presence of an antigen in the sample acts to reduce the amount of antibody against the antigen available for binding to the well and thus reduces the ultimate signal. This is also appropriate for detecting antibodies against an antigen in an unknown sample, where the unlabeled antibodies bind to the antigen-coated wells and also reduces the amount of antigen available to bind the labeled antibodies.

“Under conditions effective to allow immune complex (antigen/antibody) formation” means that the conditions preferably include diluting the antigens and/or antibodies with solutions such as BSA, bovine gamma globulin (BGG) or phosphate buffered saline (PBS)/Tween. These added agents also tend to assist in the reduction of nonspecific background. The “suitable” conditions also mean that the incubation is at a temperature or for a period of time sufficient to allow effective binding. Incubation steps are typically from about 1 to 2 to 4 hours or so, at temperatures preferably on the order of 25° C. to 27° C., or may be overnight at about 4° C. or so.

The antibodies of the present invention may also be used in conjunction with both fresh-frozen and/or formalin-fixed, paraffin-embedded tissue blocks prepared for study by immunohistochemistry (IHC). The method of preparing tissue blocks from these particulate specimens has been successfully used in previous IHC studies of various prognostic factors, and/or is well known to those of skill in the art (Brown et al., 1990; Abbondanzo et al., 1999; Allred et al., 1990).

Also contemplated in the present invention is the use of immunohistochemistry. This approach uses antibodies to detect and quantify antigens in intact tissue samples. Generally, frozen-sections are prepared by rehydrating frozen “pulverized” tissue at room temperature in phosphate buffered saline (PBS) in small plastic capsules; pelleting the particles by centrifugation; resuspending them in a viscous embedding medium (OCT); inverting the capsule and pelleting again by centrifugation; snap-freezing in −70° C. isopentane; cutting the plastic capsule and removing the frozen cylinder of tissue; securing the tissue cylinder on a cryostat microtome chuck; and cutting 25-50 serial sections.

Permanent-sections may be prepared by a similar method involving rehydration of the 50 mg sample in a plastic microfuge tube; pelleting; resuspending in 10% formalin for 4 hours fixation; washing/pelleting; resuspending in warm 2.5% agar; pelleting; cooling in ice water to harden the agar; removing the tissue/agar block from the tube; infiltrating and/or embedding the block in paraffin; and cutting up to 50 serial permanent sections.

In addition, by exploiting the intrinsic properties of mass and charge, mass spectrometry (MS) can resolved and confidently identified a wide variety of complex compounds, including proteins. Traditional quantitative MS has used electrospray ionization (ESI) followed by tandem MS (MS/MS) (Chen et al., 2001; Zhong et al., 2001; Wu et al., 2000) while newer quantitative methods are being developed using matrix assisted laser desorption/ionization (MALDI) followed by time of flight (TOF) MS (Bucknall et al., 2002; Mirgorodskaya et al., 2000; Gobom et al., 2000). These specific techniques are described elsewhere in this document.

VII. Screening for SERT Activity

In particular embodiments, the present invention provides a method for high throughput screening for blockers or inhibitors of the serotonin transporter. To accomplish this, a quick, inexpensive and easy assay to run is an in vitro assay. Such assays generally use isolated molecules, can be run quickly and in large numbers, thereby increasing the amount of information obtainable in a short period of time. A variety of vessels may be used to run the assays, including test tubes, plates, dishes and other surfaces such as dipsticks or beads.

One example of a cell free assay in this invention is the use of cellular extracts that comprise a neurotransmitter. These may be cell membrane preparations that comprise a neurotransmitter transporter, particularly a serotonin transporter.

Another example is a cell-binding assay. While not directly addressing function, the ability of an inhibitor or blocker to bind to a target molecule (in this case the serotonin transporter) in a specific fashion is strong evidence of a related biological effect. For example, binding of a molecule to a serotonin transporter may, in and of itself, be inhibitory, due to steric, allosteric or charge-charge interactions. The serotonin transporter protein may be either free in solution, fixed to a support, expressed in or on the surface of a cell. Either the serotonin transporter or the compound may be labeled, thereby permitting determining of binding. Usually, the target will be the labeled species, decreasing the chance that the labeling will interfere with or enhance binding. Competitive binding formats can be performed in which one of the agents is labeled, and one may measure the amount of free label versus bound label to determine the effect on binding.

A technique for high throughput screening of compounds is described in WO 84/03564. Large numbers of small peptide test compounds are synthesized on a solid substrate, such as plastic pins or some other surface. Bound polypeptide is detected by various methods.

A. Measurement of Transport

In some embodiments, the present invention provides a novel and rapid method for analysis of transport by a serotonin transporter that comprises the measurement of uptake and/or accumulation of serotonin and analogues thereof that are specifically taken up by the transporter. Typically, this is accomplished by measuring the uptake or binding of radiolabeled serotonin (e.g. [³H]serotonin) or a radiolabeled antagonist such as [³H]citalopram, [³H]paroxetine, or [¹²⁵I]RTI-55. Conventional assays involves the uptake of radiolabeled 5HT where antagonist sensitivity is measured for inhibition of serotonin accumulation or the inhibition of labeled antagonist binding to intact cells expressing SERT or to membranes from intact cells expressing SERT. Basically, cells transfected with a SERT construct are washed in assay buffer followed by a preincubation in 37° C. assay buffer containing 1.8 g/L glucose. This is followed by an incubation period, about 10 minutes, at 37° C. in the presence of [³H]-5-HT, or a radiolabeled antagonist such as [³H]citalopram, [³H]paroxetine, or [¹²⁵¹]RTI-55. Details of this assay are provided in the Examples.

1. Scintillation Proximity Assays

Measurement of transport may also be involve scintillation proximity assays, which is used to count the accumulated radiolabel on plates having scintillant embedded in them. Basically, cells are plated at 50% confluence on 0.4-μm pore size 6.5-mm Transwell cell culture filter inserts and grown for 7 days. A cell monolayer growing on the porous membrane of the cell culture filter insert effectively separates each well in the cell culture plate into two chambers. The apical membranes of epithelial cells plated on these filters faces the chamber above the cells and the basolateral membranes face the lower chamber through the filter. After one wash each of the apical (upper chamber) and basolateral (lower chamber) sides of the monolayer with PBS/Ca/Mg, the cells are incubated in PBS/Ca/Mg containing ³H-labeled substrate either in the upper or the lower chamber at 22° C. At the end of the incubation cells are washed either three times from the apical side and once from the basolateral side (when ³H-labeled substrate was present in the upper chamber) or once from the apical side and three times from the basolateral side (when substrate was present in the lower chamber). The apical side of the cells are washed by adding 0.2 ml of ice-cold PBS to the upper chamber and aspirating. The basolateral side of the cells are washed by pipetting ice-cold PBS over the bottoms of the filter inserts. After the washes, the filters with cells attached are excised from the insert cups, submerged in 3 ml of Optifluor scintillation fluid (Packard Instrument Co., Downers Grove, Ill.), and counted in a Beckman LS-3801 liquid scintillation counter. Transport assays on 48-well plates were described previously (Gu et al., 1994).

2. Voltage and Patch Clamp

The present invention also employs a means of determining the serotonin transporter activity or function by measuring the change in movement across a membrane, when the transporter is active. This may be accomplished using the voltage clamp technique, as is well known in the art, this allows the gating properties of the voltage-gated channels to be analyzed.

In short, the voltage clamp technique is a procedure whereby the transmembrane voltage of a membrane segment is rapidly set and maintained at a desired level. Once the membrane potential is controlled, the current flowing through the channels in that segment can be measured.

The patch clamp technique allows the voltage clamp technique to be applied to a small patch of membrane containing a single voltage-sensitive channel. The basic idea behind a patch clamp experiment is to isolate a patch of membrane so small that it contains a single voltage-gated channel. Once this patch of membrane is isolated, the single channel can be voltage clamped. Using this technique, the gating properties of the serotonin transporter can be characterized.

B. Other Methods of Measurement of Transport

Other methods of measurement contemplated in the present invention may involve fluorescence microscopy. This may involve the use of fluorescent substrates, some of which are contemplated to be analogs of other native neurotransmitters.

1. Microscopy

Fluorescent microscopy is used to measure transport using serotonin or analogues thereof which are fluorescent substrates for the serotonin transporter. Cells that either endogenously or exogenously express a serotonin transporter are isolated and plated on glass bottom Petri-dishes or multi-well plates that may typically be coated with poly-L-lysine or any other cell adhesive agent. Cells are typically cultured for three or more days. The culture medium is then aspirated and the cells are mounted on a Zeiss 410 confocal microscope. During the confocal measurement cells remain without buffer for approximately thirty seconds. Background autofluorescence is established by collecting images for ten seconds prior to the addition of the buffer and serotonin or analogues thereof. As serotonin or an analogue thereof has a large Stoke shift between excitation (1ₘₐₓ=488 nm) and emission maxima (1ₘₐₓ=610 nm), the argon laser is tuned to 488 nm and the emitted light filtered with a 580-630 nm band pass filter (Imax=610 nm). The substantial red shift can be exploited to reduce background auto-fluorescence produced in the absence of substrate. The gain (contrast) and offset (brightness) for the photomultiplier tube (PMT) may be set to avoid detector saturation at the higher serotonin concentrations that may be used in certain experiments. The effects of photo-bleaching on serotonin accumulation may also be determined by examining the rate of serotonin accumulation and decay at various acquisition rates. In a constant pool of serotonin, rates as high as 20 Hz (50 msec/image) can be set.

2. Fluorescence Anisotropy Measurements

To evaluate serotonin or analogues thereof binding to the surface membranes, cells expressing a serotonin transporter may be exposed to serotonin or analogues thereof with horizontal polarizer, with the polarizer rapidly switching to the vertical position. Cells may be imaged with alternating polarizations for 3 minutes to measure light intensity in the horizontal (Iₕ) and vertical (I_{V}) positions in order to calculate the anisotropy ratio, r=(I_{V}−gIₕ)/(I_{V}+2 g Iₕ). The factor g may be determined by using a half wave plate as described by Blackman et al. (1996). In this formulation, r=0.4 implies an immobile light source. Surface anisotropy can be measured at the cell circumference over 1 pixel width (0.625 mm). Cytosolic anisotropy can be measured near the center of the cell, approximately 5 pixel widths from the membrane.

3. Image Analysis

The fluorescent images may be processed using suitable software. For example, fluorescent images may be processed using MetaMorph imaging software (Universal Imaging Corporation, Downington Pa.). Fluorescent accumulation may be established by measuring the average pixel intensity of time resolved fluorescent images within a specified region identified by the DIC image. Average pixel intensity is used to normalize among cells.

4. Single Cell Fluorescence Microscopy

In some embodiments, the invention provides measurement of transporter characteristics at the single-cell level. Single-cell fluorescence microscopy provides a powerful assay to study rapid serotonin uptake kinetics from single cells.

5. Automation

The inventors further contemplate that all these methods are adaptable to high-throughput formats using robotic fluid dispensers, multi-well formats and fluorescent plate readers for the identification of serotonin transport modulators.

C. In Vivo Microdialysis

Microdialysis may be used in the present invention to monitor interstitial fluid in various body organs with respect to local metabolic changes. This technique may also be experimentally applied in humans for measurements in adipose tissue. In the present invention, the release of serotonin in the mouse brain, in response to stimuli may be analyzed using this technique.

Microdialysis procedure involves the insertion through the guide cannula of a thin, needle-like perfusable probe (CMA/12.3 mm×0.5 mm) to a depth of 3 mm in striatum beyond the end of the guide. The probe is connected beforehand with tubing to a microinjection pump (CMA-/100). The probe may be perfused at 2 μl/min with Ringer's buffer (NaCl 147 mM; KCl 3.0 mM; CaCl₂ 1.2 mM; MgCl₂ 1.0 mM) containing 5.5 mM glucose, 0.2 mM L-ascorbate, and 1 μM neostigmine bromide at pH 7.4). To achieve stable baseline readings, microdialysis may be allowed to proceed for 90 minutes prior to the collection of fractions. Fractions (20 μl) may be obtained at 10 minute intervals over a 3 hour period using a refrigerated collector (CMA170 or 200). Baseline fractions may be collected, following the drug or combination of drugs to be tested, been administered to the animal. Upon completion of the collection, each mouse may be autopsied to determine accuracy of probe placement.

VIII. Autism

Autism is a complex developmental disability that typically appears during the first three years of life. The result of a neurological disorder that affects the functioning of the brain, autism impacts the normal development of the brain in the areas of social interaction and communication skills. Children and adults with autism typically have difficulties in verbal and non-verbal communication, social interactions, and leisure or play activities.

Autism is one of five disorders coming under the umbrella of Pervasive Developmental Disorders (PDD), a category of neurological disorders characterized by “severe and pervasive impairment in several areas of development,” including social interaction and communications skills (DSM-IV-TR). The five disorders under PDD are Autistic Disorder, Asperger's Disorder, Childhood Disintegrative Disorder (CDD), Rett's Disorder, and PDD-Not Otherwise Specified (PDD-NOS). Each of these disorders has specific diagnostic criteria as outlined by the American Psychiatric Association (APA) in its Diagnostic & Statistical Manual of Mental Disorders (DSM-IV-TR).

Autism is the most common of the Pervasive Developmental Disorders, affecting an estimated 1 in 250 births (Centers for Disease Control and Prevention, 2003). This means that as many as 1.5 million Americans today are believed to have some form of autism. And that number is on the rise. Based on statistics from the U.S. Department of Education and other governmental agencies, autism is growing at a rate of 10-17 percent per year. At these rates, the ASA estimates that the prevalence of autism could reach 4 million Americans in the next decade. The overall incidence of autism is consistent around the globe, but is four times more prevalent in boys than girls. Autism knows no racial, ethnic, or social boundaries, and family income, lifestyle, and educational levels do not affect the chance of autism's occurrence.

While understanding of autism has grown tremendously since it was first described by Dr. Leo Kanner in 1943, most of the public, including many professionals in the medical, educational, and vocational fields, are still unaware of how autism affects people and how they can effectively work with individuals with autism. Contrary to popular understanding, many children and adults with autism may make eye contact, show affection, smile and laugh, and demonstrate a variety of other emotions, although in varying degrees. Like other children, they respond to their environment in both positive and negative ways.

Autism is a spectrum disorder. The symptoms and characteristics of autism can present themselves in a wide variety of combinations, from mild to severe. Although autism is defined by a certain set of behaviors, children and adults can exhibit any combination of the behaviors in any degree of severity. Two children, both with the same diagnosis, can act very differently from one another and have varying skills. Parents may hear different terms used to describe children within this spectrum, such as autistic-like, autistic tendencies, autism spectrum, high-functioning or low-functioning autism, more-abled or less-abled. More important than the term used is to understand that, whatever the diagnosis, children with autism can learn and function productively and show gains with appropriate education and treatment.

Every person with autism is an individual, and like all individuals, has a unique personality and combination of characteristics. Some individuals mildly affected may exhibit only slight delays in language and greater challenges with social interactions. The person may have difficulty initiating and/or maintaining a conversation. Communication is often described as talking at others (for example, monologue on a favorite subject that continues despite attempts by others to interject comments). People with autism process and respond to information in unique ways. In some cases, aggressive and/or self-injurious behavior may be present. Persons with autism may also exhibit some of the following traits: insistence on sameness; resistance to change; difficulty in expressing needs; uses gestures or pointing instead of words; repeating words or phrases in place of normal, responsive language; laughing, crying, showing distress for reasons not apparent to others; prefers to be alone; aloof manner; tantrums; difficulty in mixing with others; may not want to cuddle or be cuddled; little or no eye contact; unresponsive to normal teaching methods; sustained odd play; spins objects; inappropriate attachments to objects; apparent over-sensitivity or under-sensitivity to pain; no real fears of danger; noticeable physical over-activity or extreme under-activity; uneven gross/fine motor skills; not responsive to verbal cues; acts as if deaf although hearing tests in normal range.

There currently are no medical tests for diagnosing autism. An accurate diagnosis must be based on observation of the individual's communication, behavior, and developmental levels. However, because many of the behaviors associated with autism are shared by other disorders, various medical tests may be ordered to rule out or identify other possible causes of the symptoms being exhibited.

A brief observation in a single setting cannot present a true picture of an individual's abilities and behaviors. Parental (and other caregivers') input and developmental history are very important components of making an accurate diagnosis. At first glance, some persons with autism may appear to have mental retardation, a behavior disorder, problems with hearing, or even odd and eccentric behavior. To complicate matters further, these conditions can co-occur with autism. However, it is important to distinguish autism from other conditions, since an accurate diagnosis and early identification can provide the basis for building an appropriate and effective educational and treatment program.

Research indicates that early diagnosis is associated with dramatically better outcomes for individuals with autism. The earlier a child is diagnosed, the earlier the child can begin benefiting from one of the many specialized intervention approaches. The characteristic behaviors of autism spectrum disorders may or may not be apparent in infancy (18 to 24 months), but usually become obvious during early childhood (24 months to 6 years). As part of a well-baby/well-child visit, a child's doctor should do a “developmental screening”″ asking specific questions about a baby's progress. The National Institute of Child Health and Human Development (NICHD) lists these five behaviors that signal further evaluation is warranted: (1) does not babble or coo by 12 months; (2) does not gesture (point, wave, grasp) by 12 months; (3) does not say single words by 16 months; (4) does not say two-word phrases on his or her own by 24 months; and (5) has any loss of any language or social skill at any age. Having any of these five “red flags” does not mean your child has autism, but because the characteristics of the disorder vary so much, a child should have further evaluations by a multidisciplinary team that may include a neurologist, psychologist, developmental pediatrician, speech/language therapist, learning consultant, or other professionals knowledgeable about autism.

While there is no one behavioral or communications test that can detect autism, several screening instruments have been developed that are now used in diagnosing autism:
CARS rating system (Childhood Autism Rating Scale), developed by Eric Schopler in the early 1970s, is based on observed behavior. Using a 15-point scale, professionals evaluate a child's relationship to people, body use, adaptation to change, listening response, and verbal communication.
The Checklist for Autism in Toddlers (CHAT) is used to screen for autism at 18 months of age. It was developed by Simon Baron-Cohen in the early 1990s to see if autism could be detected in children as young as 18 months. The screening tool uses a short questionnaire with two sections, one prepared by the parents, the other by the child's family doctor or pediatrician.
The Autism Screening Questionnaire is a 40 item screening scale that has been used with children four and older to help evaluate communication skills and social functioning.
The Screening Test for Autism in Two-Year Olds, being developed by Wendy Stone at Vanderbilt, uses direct observations to study behavioral features in children under two. She has identified three skills areas—play, motor imitation, and joint attention—that seem to indicate autism.

While there is no cure for autism, there are treatment and education approaches that may reduce some of the challenges associated with the disability. Intervention may help to lessen disruptive behaviors, and education can teach self-help skills that allow for greater independence. But just as there is no one symptom or behavior that identifies autistic children, there is no single treatment. Children can learn to function within the confines of their disability, but treatment must be tailored to the child's individual behaviors and needs.

IX. Kits

All the essential materials and reagents required for detecting nucleic acid mutations or proteins in a sample may be assembled together in a kit. This generally will comprise a primer or probe designed to hybridize specifically to or upstream of target nucleotides of the polymorphism of interest, or a protein binding agents, e.g., and antibody. The primer, probe or antibody may be labeled with a radioisotope, a fluorophore, a chromophore, a dye, an enzyme, or TOF carrier. Also included may be enzymes suitable for amplifying nucleic acids, including various polymerases (reverse transcriptase, Taq, etc.), dNTPs/rNTPs and buffers (e.g., 10× buffer=100 mM Tris-HCl (pH 8.3), and 500 mM KCl) to provide the necessary reaction mixture for amplification. One or more of the deoxynucleotides may be labeled with a radioisotope, a fluorophore, a chromophore, a dye, or an enzyme. Such kits may also include enzymes and other reagents suitable for detection of specific nucleic acids or amplification products.

The container means of the kits will generally include at least one vial, test tube, flask, bottle, or other container means, into which a component may be placed, and preferably, suitably aliquoted. Where there is more than one component in the kit, the kit also will generally contain additional containers into which the additional components may be separately placed. However, various combinations of components may be comprised in a container. The kits of the present invention also will typically include a means for packaging the component containers in close confinement for commercial sale. Such packaging may include injection or blow-molded plastic containers into which the desired component containers are retained.

X. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

### Materials and Methods

Sample and Genetic Analyses. The sample for this study consisted of 73 families recruited from Tufts-Vanderbilt consortium and 267 from the AGRE consortium (Table 1). The demographics of these populations have been reported previously (Yonan et al., 2003; McCauley et al., 2005), as have diagnostic inclusion and exclusion criteria for linkage analysis (McCauley et al., 2005). Families were selected based on there being a single proband who met full ADI criteria for autism and a second sibling who was diagnosed with autism or presented on the broader spectrum. In the current report, the inventors further examine linkage in a larger sample of 341 families with at least two affected children; for association studies and screening for known variants (e.g., Gly56Ala) we included an additional 43 trios (both parents and the proband). As reported previously (McCauley et al., 2005), Tufts/Vanderbilt families were genotyped at deCODE (Reykjavik, Iceland) using their 500 marker panel with an average inter-marker spacing of ˜8 cM. Two-point and multipoint heterogeneity LOD (HLOD) scores on individual and combined samples, respectively, were calculated under both dominant and recessive models using Allegro (Gudbjartsson et al., 2000). Disease allele frequency was estimated to be 0.01 and 0.1 for dominant and recessive models, respectively. Phenotypic status was only considered for affected individuals, and other family members were designated as having an unknown phenotypic status. Nonparametric allele-sharing LOD* values were calculated using affected relative pair data based on an exponential model using the Spairs scoring function as recommended by McPeek (McPeek, 1999). NPL scores and corresponding P-values were also calculated by Allegro. The position of chromosome 17 markers is based on the deCODE genetic map (Kong et al., 2002).

TABLE 1
Autism Family Samples for Linkage and Association Studies

| | Multiplex Families | | | Trio Families | | | |
|---|---|---|---|---|---|---|---|
| Site | MO | FC | Total | MO | FC | Total | Totals |
| AGRE | 156 | 111 | 267 | 11 | 5 | 16 | 283 |
| Tufts/VU | 46 | 28 | 74 | 22 | 5 | 27 | 101 |
| Total | 202 | 139 | 341 | 33 | 10 | 43 | 384 |

Association tests of 5-HTTLPR and rs140700 were performed using the Pedigree Disequilibrium Test (PDT) statistic (Martin et al., 2000), a variant of the transmission disequilibrium test (TDT), developed for use with general pedigrees. Genotype analysis of these markers has been reported previously (McCauley et al., 2004). Other tests of association or comparison of allele frequencies involved generation of a c2 statistic and corresponding P-values using standard 2×2 contingency tables. Comparison of male to female affection for autism in the presence of Gly56Ala alleles was performed using Fisher's Exact test. Comparison of ADI-derived variable cluster scores was performed using the T-test, incorporating subject numbers, means and standard deviations from the overall dataset and either individuals within a given family, or family means across multiple families to evaluate the significance of score differences. Significance is reported as two-tailed P-values. Approval for these studies was granted by the respective Institutional Review Boards at Tufts University School of Medicine/New England Medical Center and Vanderbilt University Medical Center. All studies were performed with informed consent provided by the families participating in the research.

Variant Discovery. Known but rare SLC6A4 coding variants reported in previous studies (Cargill et al., 1999; Glatt et al., 2001; Hahn and Blakely 2002; Ozaki et al., 2003) were screened in 327 multiplex and 57 parent-child trio families using TaqMan™ allelic discrimination assays (Table 2). Where possible, variants were assayed within exons to permit inclusion of in vitro mutagenized plasmid cDNA samples alone, or mixed with wild-type cDNAs, to provide homozygous and heterozygous controls, respectively. ABI TaqMan™ (Holloway et al., 1999) reactions were performed in a 5 μl volume according to manufacturer's recommendations (Applied Biosystems). Cycling conditions included an initial denaturation at 95° C. for 10 min, followed by 50 cycles of 92° C. for 15 s, 60° C. for 1 min. Samples were analyzed using an ABI 7900HT Sequence Detection System. Screening for unknown variants involved arbitrary selection of one affected individual from each of 120 families, ranked by family-specific nonparametric LOD * scores from the overall 340-family dataset.

TABLE 2

SLC64A SNP Marker Information

| | | | | | |
|---|---|---|---|---|---|
| | | | Inter- | | Pro- |
| Mar- | Mar- | dbSNP rs# | marker | Forward/Reverse | duct |

| | | | | | | | | | | |
|---|---|---|---|---|---|---|---|---|---|---|
| ker | ker | SLC6A4 | Celera | | | Distance | | Probes | Size | As- |
| No. | Type | Region | hCV8 | Alleles | MAF | (bp) | Primers | VIC/FAM | (bp) | say |

| | | | | | | | | | | |
|---|---|---|---|---|---|---|---|---|---|---|
| | t | | rs1050565 | | | | N/A | N/A | t | |
| 1 | SNP | Promoter 1A | | T/C | 0.32 | 10,035 | | | N/A | AoD |
| | | | hCV7473213 | | | | | | | |
| 2 | SNP | Promoter 1A | rs2020930 | G/A | 0.03 | 1,734 | 5′- | 5′-VIC | 72 | AED |
| | | | | | | | GCTCAAGCAGGTGAACAA | AACTATTGCTATGCGGTGAT- | | |
| | | | | | | | AGAAA-3′ | MGB-NFQ-3′ | | |
| | | | | | | | (SEQ ID NO: 3) | (SEQ ID NO: 41) | | |
| | | | hCV11424041 | | | | 5′- | 5′-FAM- | | |
| | | | | | | | CTGGGCAGCTGGGAAGAG- | TTGCTGTGCGGTGAT- | | |
| | | | | | | | 3′ | MGB-NFQ-3′ | | |
| | | | | | | | (SEQ ID NO: 4) | (SEQ ID NO: 42) | | |
| 3 | INS/ | Promoter 1A | 5-HTTLPR | 528(L) | 0.44 | 2,549 | 5′- | | 484 | Gel |
| | | | | | | | GGCGTTGCCGCTCTGAATG | | | |
| | | | | | | | C-3′ | | | |
| | | | | | | | (SEQ ID NO: 5) | | | |
| | DEL | | | 484(5) | | | 5′- | | 528 | |
| | | | | | | | GAGGGACTGAGCTGGACA | | | |
| | | | | | | | ACCAC-3′ | | | |
| | | | | | | | (SEQ ID NO: 6) | | | |
| | | | rs2020933 | | | | 5′- | 5′-VIC- | | |
| | | | | | | | TGTATGTATTTTTACCATC | CATTGACCAGGTTCAC- | | |
| | | | | | | | AGTTTTGTCCAGAA-3′ | MGB-NFQ-3′ | | |
| | | | | | | | (SEQ ID NO: 7) | (SEQ ID NO: 43) | | |
| 4 | SNP | Intron 1A | hCV11424045 | A/T | 0.07 | 295 | 5′- | 5′-FAM- | 81 | AbD |
| | | | | | | | GAGAGTTAGCTAGCAGGC | CATTGACCTGGTTCAC- | | |
| | | | | | | | TCATAAAT-3′ | MGB-NFQ-3′ | | |
| | | | | | | | (SEQ ID NO: 8) | (SEQ ID NO: 44) | | |
| | | | rs2020934 | | | | 5′-TTTTCCTG CCACG | 5′-VIC- | | |
| | | | | | | | CACTCT-3′ | ACCGTTCCAATATGG- | | |
| | | | | | | | (SEQ ID NO: 9) | MGB-NFQ-3′ | | |
| | | | | | | | | (SEQ ID NO: 45) | | |
| 5 | SNP | Intron 1A | hCV7911197 | C/T | 0.47 | 5 | 5′- | 5′-FAM- | 80 | AbD |
| | | | | | | | GCACAAACCTCATAAGAA | CCGTTCCAACATGG- | | |
| | | | | | | | CCTGCTT-3′ | MBG-NFQ-3 | | |
| | | | | | | | (SEQ ID NO: 10) | (SEQ ID NO: 46) | | |
| | | | rs2020935 | | | | 5′-TG GCAGTGACCG | 5′-VIC- | | |
| | | | | | | | TTCCAA-3′ | CTGCTTCTCACTCATCCA- | | |
| | | | | | | | (SEQ ID NO: 11) MGB-NFQ-3′ | | | |
| | | | | | | | | (SEQ ID NO: 47) | | |
| 6 | SNP | Intron 1A | hCV11424046 | T/A | 0.07 | 11,477 | 5′- | 5′-FAM- | 68 | AbD |
| | | | | | | | TTGCTCAATTTGCACAAAC | TGCTTCTCACTCAACCA- | | |
| | | | | | | | CTCAT-3′ | MGB-NFQ-3′ | | |
| | | | | | | | (SEQ ID NO: 12) | (SEQ ID NO: 48) | | |
| | | | rs25528 | | | | 5′-C CCAG T G G AG | 5′-VIC- | | |
| | | | | | | | G CACAG G-3′ | TGGTTGGTGTCGCCG- | | |
| | | | | | | | (SEQ ID NO: 13) | MGB-NFQ-3′ | | |
| | | | | | | | | (SEQ ID NO: 49) | | |
| 7 | SNP | Intron 1A | hCV1841705 | A/C | 0.16 | 80 | 5′-GAGTGTG CAG G | 5′-FAM- | 62 | AbD |
| | | | | | | | TTACTGATG CT-3′ | TGGTTGGTTTCGCCG- | | |
| | | | | | | | (SEQ ID NO: 14) | MGB-NFQ-3′ | | |
| | | | | | | | | (SEQ ID NO: 50) | | |
| | | | rs6354 | | | | 5′-G G AG G CAAG G | 5′-VIC- | | |
| | | | | | | | C G ACCTT-3′ | CTTGCCCTCTATTGCAG- | | |
| | | | | | | | (SEQ ID NO: 15) | MGB-NFQ-3′ | | |
| | | | | | | | | (SEQ ID NO: 51) | | |
| 8 | SNP | Exon tB | hCV184t706 | A/C | 0.17 | 931 | 5′-CTGTG GCTAAGC | 5′-FAM- | 58 | AbD |
| | | | | | | | CCCTTGTTATT-3′ | TTGCCCTCTCTTGCAG- | | |
| | | | | | | | (SEQ ID NO: 16) | MGB-NFQ-3′ | | |
| | | | | | | | 5′- | (SEQ ID NO: 52) | | |
| | | | | | | | GTCATTTACTAACCAGCAG | 5′-VIC- | | |
| | | | | | | | GATGGA-3′ | ATTCAAGGGCGTCGTC- | | |
| | | | | | | | (SEQ ID NO: 17) | MGB-NFQ-3′ | | |
| | | | | | | | | (SEQ ID NO: 53) | | |
| 9 | SNP | Exon 2 | Thr4Ala | A/G | | 157 | 5′- | 5′-FAM- | 62 | AbO |
| | | | | | | | CGCTGATAGCTGCTTCTGA | CAAGGGCGCCGTC- | | |
| | | | | | | | GA-3′ | MGB-NFQ-3′ | | |
| | | | | | | | (SEQ ID NO: 18 | (SEQ ID NO: 54) | | |
| | | | | | | | 5′-G G GTACTCAG CAG | 5′-VIC- | | |
| | | | | | | | TTCCAAGTC-3′ | CTGGTGCGGGAGAT- | | |
| | | | | | | | (SEQ ID NO: 19 | MGB-NFQ-3′ | | |
| | | | | | | | | (SEQ ID NO: 55) | | |
| 10 | SNP | Exon 2 | Gly56Ala | G/C | 0.01 | 310 | 5′-G GGATAGAGTG CCG | 5′-FAM- | 56 | AbD |
| | | | | | | | TGTG T-3′ | CTGGTGCGGCAGAT- | | |
| | | | | | | | (SEQ ID NO: 20) | GB-NFQ-3′ | | |
| | | | | | | | | (SEQ ID NO: 56) | | |
| | | | | 9 | 0.02 | | 5′- | | | |
| | | | | | | | TGGATTTCCTTCTCTCAGT | | | |
| | | | | | | | GATTGG-3′ | | | |
| | | | | | | | (SEQ ID NO: 21) | | | |
| 11 | VNTR | Intron 2 | VNTR | 10 | 0.39 | 3,269 | 5′- | | 345 | Gel |
| | | | | | | | TCATGTTCCTAGTCTTACG | | 360 | |
| | | | | | | | CCAGTG-3′ | | | |
| | | | | | | | (SEQ ID NO: 22) | | | |
| | | | | 12 | 0.59 | | 5′- | 5′-VIC- | 390 | |
| | | | | | | | GCTATACTACCTCATCTCC | CCAGGAGTTCTTGCAGC- | | |
| | | | | | | | TCCTTCAC-3′ | MGB-NFQ-3′ | | |
| | | | | | | | (SEQ ID NO: 23) | (SEQ ID NO: 57) | | |
| 12 | SNP | Exon 4 | Lys201Asn | G/C | | 40 | 5′-TG GTGCAG TTG | 5′-FAM- | 83 | AbD |
| | | | | | | | CCAGTGTT-3′ | CAGGAGTTGTTGCAGC- | | |
| | | | | | | | (SEQ ID NO: 24) | MGB-NFQ-3′ | | |
| | | | | | | | 5′- | (SEQ ID NO: 58) | | |
| | | | | | | | TCCTGGAACACTGGCAACT | 5′-VIC- | | |
| | | | | | | | G-3′ | AATTACTTCTCCGAGGACAA- | | |
| | | | | | | | (SEQ ID NO: 25) | MGB-NFQ-3′ | | |
| | | | | | | | | (SEQ ID NO: 59) | | |
| 13 | SNP | Exon 4 | Glu215Lys | G/A | | 1,802 | 5′-GAATG GAG G | 5′-FAM- | 65 | AbD |
| | | | | | | | GTCCAG GTGATG-3′ | AATTACTTCTCCAAGGACAA- | | |
| | | | | | | | (SEQ ID NO: 26) | MGB-NFQ-3′ | | |
| | | | | | | | | (SEQ ID NO: 60) | | |
| | | | rs140700 | | | | 5′- | 5′-VIC- | | |
| | | | | | | | ACTCCAAGGGTTGTGATCT | ACCACCTCACCCTCCT- | | |
| | | | | | | | TTCTG-3′ | MGB-NFQ-3′ | | |
| | | | | | | | (SEQ ID NO: 27) | (SEQ ID NO: 61) | | |
| 14 | SNP | Intron5 | hCV7473202 | G/A | 0.07 | 195 | 5′-GGGTGAATG | 5′-FAM- | 89 | AbD |
| | | | | | | | GATGTCAGTGTCTTTT-3′ | CACCTCGCCCTCCT- | | |
| | | | | | | | (SEQ ID NO: 28) | MGB-NFQ-3′ | | |
| | | | | | | | 5′- | (SEQ ID NO: 62) | | |
| | | | | | | | GTGACAGCCACCTTCCCTT | 5′-VIC- | | |
| | | | | | | | ATATC-3′ | CAGGACAGAAAGGAT- | | |
| | | | | | | | (SEQ ID NO: 29) | MGB-NFQ-3′63) | | |
| 15 | SNP | Exon 6 | Ser293Phe | C/T | | 505 | 5′- | 5′-FAM- | 56 | AbD |
| | | | | | | | GTGGCACCCCTCACCAG- | CAGGACAAAAAGGAT- | | |
| | | | | | | | 3′ | MGB-NFQ-3′ | | |
| | | | | | | | (SEQ ID NO: 30) | (SEQ ID NO: 64) | | |
| | | | | | | | 5′- | 5′-VIC- | | |
| | | | | | | | AGCCGCTCAGATCTTCTTC | CAAAGCCCGGAGCAA- | | |
| | | | | | | | TCT-3′ | MGB-NFQ-3′ | | |
| | | | | | | | (SEQ ID NO: 31) | (SEQ ID NO: 65) | | |
| 16 | SNP | Exon 7 | Pro339Leu | C/T | | 2,811 | 5′- | 5″-FAM- | 75 | AbD |
| | | | | | | | TTGAACTTGTTGTAGCTAG | CAAAGCCCAGACCRA- | | |
| | | | | | | | CAAAAGC-3′ | MGB-NFQ-3′ | | |
| | | | | | | | (SEQ ID NO: 32) | (SEQ ID NO: 66) | | |
| | | | | | | | 5′-C G G C CCC TTG | 5′-VIC- | | |
| | | | | | | | G G TTTTC-3′ | CCAGAGATGCCCTGGTGA- | | |
| | | | | | | | (SEQ ID NO: 33) | MGB-NFQ-3′ | | |
| | | | | | | | | (SEQ ID NO: 67) | | |
| 17 | SNP | Exon 8 | Leu362 Met | C/A | | 1,504 | 5′-GAAG CTCG TCATG | 5-FAM- | 68 | AbD |
| | | | | | | | CAG TTCAC-3′ | CAGAGATGCCATGGTGA- | | |
| | | | | | | | (SEQ ID NO: 34) | MGB-FQ-3′ | | |
| | | | | | | | 5′-G CAG AAG C G | (SEQ ID NO: 68) | | |
| | | | | | | | ATAG C CAACAT G-3′ | 5′-VIC- | | |
| | | | | | | | (SEQ ID N: 35) | TTTCTTTGCCATCATCTT- | | |
| | | | | | | | | MGB-NFQ-3′ | | |
| | | | | | | | | (SEQ ID NO: 69) | | |
| 18 | SNP | Exon 9 | Ile425Val | A/G | | 8,181 | 5′-CAAGCCCAGCG TG | 5′-VIC- | 78 | AbD |
| | | | | | | | ATTAACATC-3′ | TCTTTGCCGTCATCTT- | | |
| | | | | | | | (SEQ ID NO: 36) | MGB-NFQ-3′ | | |
| | | | | | | | 5′- | (SEQ ID NO: 70) | | |
| | | | | | | | TCCCCACATATATAGCTTA | 5′-VIC- | | |
| | | | | | | | TCG G TTGA-3′ | CACGTACCTCTTTAAAT- | | |
| | | | | | | | (SEQ ID NO: 37) | MGB-NFQ-3′ | | |
| | | | | | | | | (SEQ ID NO: 71) | | |
| 19 | SNP | Exon 13 | Lys605Asn | A/C | | 4,687 | 5′- | 5′-FAM- | 105 | AbD |
| | | | | | | | CAAAACAATTAGTAGTCT | ACGTACCTCGTTAAAT- | | |
| | | | | | | | GAACACACACA-3′ | MGB-NFQ-3′ | | |
| | | | | | | | (SEQ ID NO: 38) | (SEQ ID NO: 72) | | |
| | | | | | | | 5′- | 5′-VIC- | | |
| | | | | | | | GCGTATTATTAAAAGTATT | CACAAGGAATTTCT- | | |
| | | | | | | | ACCCCAGAAACAC-3′ | MGB-NFQ-3′ | | |
| | | | | | | | (SEQ IN NO: 39) | (SEQ ID NO: 73) | | |
| 20 | SNP | Exon 14 | Pro621Ser | C/T | | — | 5′-CACAGCATTCAAGCG | 5′-FAM- | 73 | AbD |
| | | | | | | | GATGTC-3′ | CACAAGAAATTTCT- | | |
| | | | | | | | (SEQ ID NO: 40) | MGB-NFQ-3′ | | |
| | | | | | | | | (SEQ ID NO: 74) | | |

Screening of PCR products for all exons was performed on the first 24 samples using temperature gradient capillary electrophoresis (TGCE) (Li et al., 2002) on a 96-capillary Reveal™ system according to manufacturer's recommendations (Spectrumedix). Putative variants detected using TGCE were confirmed by direct sequencing of PCR product using ABI dye terminators in the Center for Molecular Neuroscience Neurogenomics Core. The promoter for the initial 24 individuals, and both exons and promoter for samples from one proband each from the remaining 96 (of 120) families were analyzed for variation by double-stranded direct sequencing of PCR products using ABI dye terminator chemistry. ABI electropherogram data obtained from Vanderbilt Cores or from Polymorphic DNA Technologies were imported and analyzed for variation using the Phred/Phrap/Consed and PolyPhred suite of sequence analysis tools (Nickerson et al., 1997; Gordon et al., 2001; Nickerson et al., 2001). Variant confirmation and segregation of rare variants was determined by sequencing available family members and the original proband in the same manner. Location of variation within the gene was documented in Table 2 using nomenclature as described previously (den Dunnen and Antonarakis, 2001).

Functional Analysis of Gly56Ala Activity and Regulation. EBV-transformed lymphocyte cell lines from AGRE or Tufts/Vanderbilt autism families carrying either the Gly56 or 56Ala alleles were obtained from the NIMH Human Genetics Initiative Repository (www.nimhgenetics.org/) at Rutgers University. Lymphocytes were cultured in suspension in RPMI 1640 medium (supplemented with 15% fetal bovine serum, 2 mM L-glutamine, 100 units/ml penicillin, and 100 μg/ml streptomycin) at 37° C. in a humidified incubator at 5% CO₂ prior to assay. Lymphocytes were pelleted at 1500 rpm, 5 min and washed with KRH assay buffer. A total of 1×10⁶ cells in triplicate were pre-warmed (37° C.) in a shaking water bath (10 min) in 12×75 polypropylene tubes in KRH buffer containing 100 μM pargyline and 100 μM ascorbic acid. After a 5-min incubation with [³H]5-HT (20 nM) at 37° C., uptake assays were terminated by immersion on ice, and uptake in pelleted, 1% SDS-extracted cells quantitated by scintillation spectrometry. Specific 5-HT uptake was determined by subtracting the amount of [³H]5-HT accumulated in the presence of 10 μM paroxetine (SmithKline Beecham). [³H]L-glutamate transport assays were conducted as described for [³H]5-HT except using 100 nM substrate and defining nonspecific uptake with parallel accumulation acquired at 4° C.

### EXAMPLE 2

### Results

Linkage. Based on initial linkage results (McCauley et al., 2004) and other reports of linkage to 17q (International Molecular Genetic Study of Autism Consortium 2001; Yonan et al., 2003), the inventors substantially increased samples of 73 Tufts/Vanderbilt and 85 AGRE families through acquisition from the NIMH Human Genetics Initiative Repository. Then, they we reassessed evidence for linkage in the larger sample containing 182 additional AGRE families. A striking linkage was observed in an overall dataset of 340 multiplex families with peak recessive heterogeneity LOD (HLODREC) score of 5.8 (FIG. 1A) at two adjacent markers at ˜53 cM (D17S1800, ˜1.4 Mb from SLC6A4 and D17S1294, ˜150 kb from SLC6A4). Results from all parametric and nonparametric linkage analyses are detailed in Table 3. Influenced by two recent reports (Stone et al., 2004; Weiss et al., 2005), we incorporated a sex-specific approach in our linkage analysis of chromosome 17. We queried for male-specific effects by splitting the dataset into (1) families containing only affected males (MO), and (2) the remaining female-containing (FC) families, similar to the approach of Stone and colleagues (Stone et al., 2004). Linkage increased substantially in the MO sample (HLODREC=8.0; n=202), with negligible contribution from the 138 remaining FC families. The magnitude of linkage overall and in the MO families, extraordinary for a complex behavioral disorder, encouraged the inventors to pursue more detailed studies to identify autism-associated alleles within SLC6A4.

TABLE 3
Autism Linkage Data for Chromosome 17

| | | | HLOD | HLOD | | LOD* | | NPL |
|---|---|---|---|---|---|---|---|---|
| Families | Marker | cM¹ | (Dom)² | (Rec)² | LOD* | P-value³ | NPL | P-value³ |
| Overall (340) | D17S1800 | 53 | 5.00 | 5.81 | 5.80 | 6.14 × 10⁻⁷ | 4.88 | 1.70 × 10⁻⁷ |
| Male-Only (MO; 189)⁴ | D17S1800 | 53 | 5.27 | 7.98 | 6.65 | 4.85 × 10⁻⁸ | 5.18 | 1.46 × 10⁻⁸ |
| Female-Containing (FC; 38)⁵ | D17S1800 | 53 | 0.38 | 0.10 | 0.45 | 0.085 | 1.37 | 0.076 |

Note
¹Centimorgans; position on the deCODE chromosome 17 genetic map
²Dominant (Dorn) or Recessive (Rec) heterogeneity multipoint LOD (HLOD) scores
³P-values are nominal and not corrected
⁴Families with only affected males
⁵Families with one or more affected females

Association. Initially, the inventors tested for evidence of allelic association using the two markers (5-HTTLPR and the intron 5 SNP rs140700), that previously demonstrated modest association in a smaller sample (McCauley et al., 2004). In a sample of 384 combined multiplex and trio families, we detected evidence for allelic association to autism (i.e., transmissions [T]>non-transmissions [NT]) only at the intron 5 marker rs140700 (minor allele T:NT=93:124; c2=4.47; P=0.03). However, when MO and FC subsets were examined separately, modest association of the S allele of 5-HTTLPR was detected in the MO dataset (N=235 families; T:NT=362:314; c2=4.90; P=0.03) as well as persistent under-transmission of the minor allele at rs140700 (T:NT=47:76; c2=6.84; P=0.009), while no association or trend towards association was seen in the 149 FC families (HTTLPRS:T:NT=223:231, c2=0.07, P=0.79; rs140700: T:NT=46:48, c2=0.021, P=0.88). The association in MO families is consistent with previous results (McCauley et al., 2004), and the male bias in linkage (Stone et al., 2004), but fails to explain the highly significant linkage in MO families shown above.

Allelic Heterogeneity at SLC6A4. Since common alleles across the SLC6A4 locus do not explain the observed linkage, we considered the hypothesis that multiple, possibly rare, SLC6A4 risk alleles exist and confer risk to autism. The inventors tested their hypothesis using two parallel strategies. First, all families were screened for known rare coding variants to determine if one or more is present at elevated frequency in our autism sample, and by inference potentially related to disease risk. The second approach involved selecting unrelated probands from multiplex families most contributing to linkage and minimally having a positive LOD score at the linkage peak and screening their SLC6A4 exons and promoter sequences for novel variants. In the combined sample of 384 multiplex and parent-child trio families, the inventors screened for known nonsynonymous variants using TaqMan™ allelic discrimination assays, and they detected multiple individuals carrying one or two copies of the 56Ala-encoding allele and a single subject heterozygous for an allele encoding the nonsynonymous variant Lys605Asn.

Detailed analysis of families with the Gly56Ala substitution reveal its presence in both “linked” (positive LOD scores) and “unlinked” multiplex families and trios. Within the 120 families with the highest family-specific LOD scores, the 56Ala allele was found to be present on 11 of 480 independent chromosomes (2.3%). Three homozygous 56Ala subjects were identified in these families. In contrast, Glatt et al. (2001) reported the 56Ala variant occurred on only 4 of 900 chromosomes in the only large non-clinical comparison sample described, with a minor allele frequency (MAF) of 0.44% and no homozygous subjects. In the remaining autism families, a lower frequency was found for the 56Ala allele with 12 of 1056 chromosomes (1.1%) harboring the variant, and here no homozygotes were identified. An alternative comparison comes from the screening of a predominantly Caucasian population ascertained for Axis I Mood Disorders. Here, the inventors identified 3 of 272 chromosomes (1.1%) with 56Ala alleles (Field, Prasad, Blakely, Sanders-Bush and Shelton, unpublished findings).

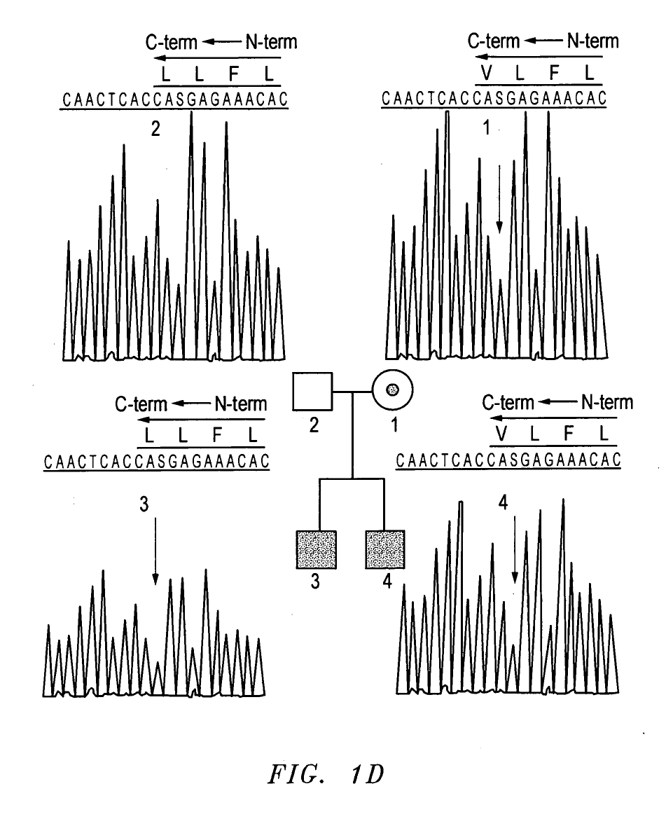

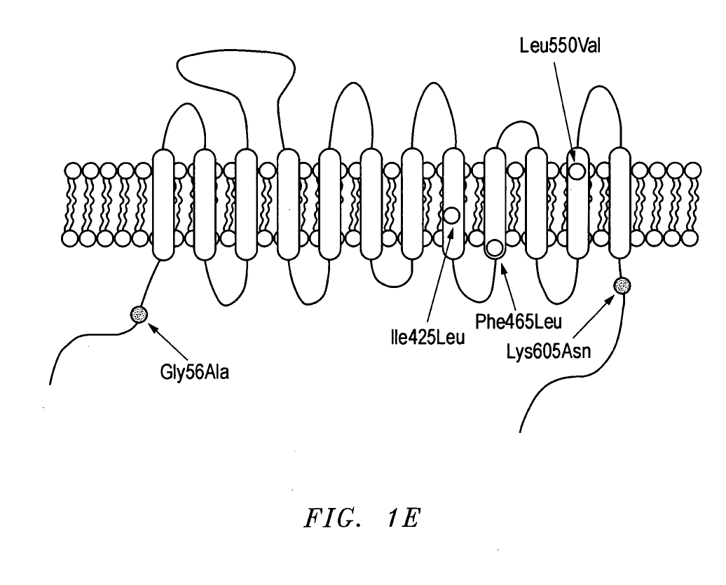

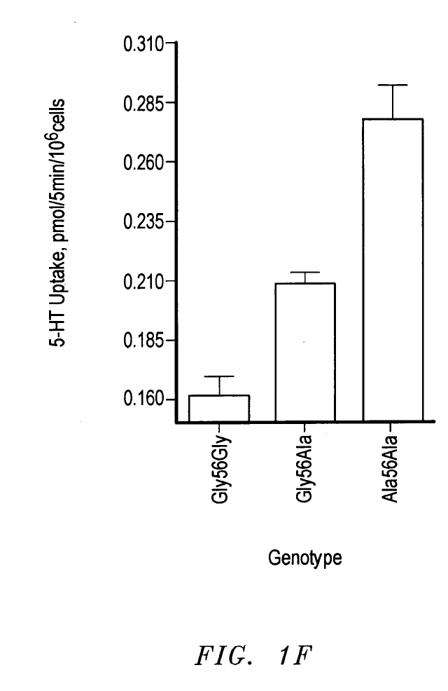

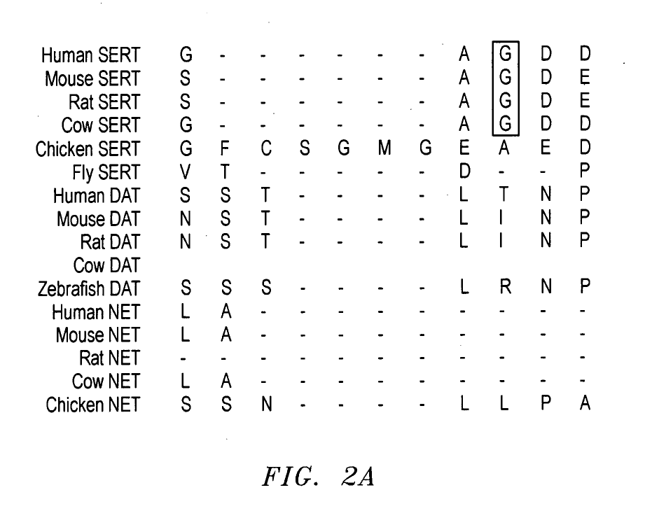

A parallel strategy involved screening the promoter, exons, and flanking sequence, in unrelated probands from, initially, 24 families most contributing to linkage in this region, based on ranking for family-specific LOD scores. Temperature gradient capillary electrophoresis (TGCE), followed by direct sequencing of PCR products comprised the initial effort, while one proband sample each from the remaining 96 (of 120) families was subjected to direct sequencing of PCR products. In addition to the independent identification of one 56Ala homozygote, two novel coding variants (Ile425Leu and Leu550Val; FIGS. 1B and 1D) were detected in the 24 most linked families (4/48=8.3% allele frequency). In the remaining 96 families, several 56Ala alleles and a Phe465Leu nonsynonymous substitution (FIG. 1C) was detected (Table 4). All three coding substitutions occur within transmembrane domains (FIG. 1E), and are highly conserved (FIGS. 2A-B). The least conserved of the novel variants corresponds to residue Ile425, which is present within all SERT proteins from human to Drosophila. The other two nonsynonymous variants occur at residues conserved from Homo sapiens to Drosophila in all monoamine transporters including SERT, the norepinephrine transporter (NET) and the dopamine transporter (DAT), for which sequence was available.

TABLE 4
Transmission Disequilibrium of Multiple Coding and Noncoding Alleles
at SLC6A4

| Location | I.D | Position¹ | Protein | Families | T² | T³ | NT | AUT♂ | AUT♀ |
|---|---|---|---|---|---|---|---|---|---|
| Exon 1b | ss38318598 | c.147C>A | 5′ UTR | 2 | 4 | 2 | 1 | 3 | 1 |
| Exon 2 | rs6355 | c.462G>C | GIy56Ala⁴ | 7 | 15 | 8 | 5 | 11 | 4 |
| Exon 9 | ss38318597 | c.1568A>C | Ile425Leu | 1 | 2 | 1 | 0 | 2 | 0 |
| Exon 10 | ss38318600 | c.1688T>C | Phe465Leu | 1 | 2 | 1 | 1 | 1 | 1 |
| Exon 12 | ss38318601 | c.1943G>C | Leu550Val | 1 | 2 | 1 | 0 | 2 | 0 |
| Exon 13 | Glatt et al | c.2110A>C | Lys605Asn | 1 | 0 | 0 | 2 | 0 | 0 |
| Exon 14 | rs13306796 | c.2516A>G | 3′ UTR | 1 | 2 | 1 | 0 | 2 | 0 |
| Promoter | ss38318589 | g.−15622G>A | N/A | 1 | 1 | 0 | 1 | 1 | 0 |
| Promoter | rs2020932 | g.−14519A>T | N/A | 3 | 8 | 5 | 0 | 6 | 0 |
| Promoter | 5538318590 | g.−14289A>C | N/A | 15 | 1 | 0 | 1 | 1 | 0 |
| Promoter | rs25533 | g.−13912T>C | N/A | 1 | 4 | 3 | 0 | 2 | 0 |
| Promoter | ss38318591 | g.−13754C>T | N/A | (1)⁶ | (1) | (0) | (1) | (1) | (0) |
| Intron 1a | ss38318592 | IVS1a+20C>T | N/A | 1 | 1 | 0 | 0 | 1 | 0 |
| Intron 1a | ss38318593 | IVS1a+133G>A | N/A | 1 | 1 | 0 | 0 | 1 | 0 |
| Intron 1 a | hcv11414117 | IVS1 a−47G>C | N/A | 5 | 10 | 5 | 2 | 8 | 2 |
| Intron 1a | hcv11414114 | IVS1a−25G>A | N/A | 4 | 9 | 5 | 0 | 7 | 2 |
| Intron 1b | ss38318594 | IVS1b+28G>A | GIy56Ala⁷ | 5 | N/A | N/A | N/A | N/A | N/A |
| Intron 6 | ss38318595 | IVS6−44G>C | N/A | 1 | 2 | 1 | 0 | 2 | 0 |
| Intron 7 | ss38318596 | IVS7+83C>T | N/A | 4 | 9 | 5 | 0 | 9 | 2 |
| Intron 8 | ss38318597 | IVS8−33C>T | N/A | 2 | 4 | 2 | 0 | 4 | 0 |

| | | | | | | |
|---|---|---|---|---|---|---|
| T^{2:} NT = 76:12x² = 26.82; 1 df; P = 2.2 × 10⁷ | 31 | 76 | 40 | 12 | 63 | 12 |
| T³:NT = 39:12x² = 8.13; 1 df; P = 0.0042 | | | | | | |

Note
¹Changes in the cDNA are indicated relative to the SLC6A4 Reference Sequence (NM_001045); Genomic variants are deisgnated by +1 corresponding to the initiating ATG or position within an intron
²Transmissions to all affected individuals, including the proband in whom the variant was first identified
³Transmissions excluding the screened proband in whom the variant was initially identified
⁴Four 56A1a NT derive from two dual heterozygous couples transmitting only one 56A1a allele to affected children
⁵Redundant transmissions are not counted towards total transmissions
⁶Family does not contribute to linkage and corresponding counts are not included in the totals.
⁷IVS7b+28G>A lies on the GIy56Ala allele, therefore transmission was not considered to avoid redundancy

To consider the genetic relevance of the coding variants to autism risk, we asked if these variants segregated with disease in each family. Two of the three novel coding variants were transmitted to all affected individuals (5 males, 1 female) in the three families (FIGS. 1B-D). One affected male did not inherit the paternally-transmitted Phe465Leu variant allele. The 425Leu allele exhibited a particularly intriguing overall segregation pattern (FIG. 1B). This variant was maternally transmitted to both affected sons. Three unaffected daughters also inherited this allele, but not two other unaffected sibs, one male and one female. Considering the clear sex-bias in linkage, segregation of the 425Leu allele in this family is consistent with, but not proof of, male-biased genetic risk or elevated penetrance associated with the allele. No unaffected siblings were present in the other two families. The 56Ala allele was detected in seven linked families, in which the transmission to non-transmission ratio (T:NT) was 15:5 (Table 2). Of the five non-transmissions, four correspond to 2 distinct instances in which both parents were carriers (expected to occur in 1 of every 2000 couples, under the assumption of Hardy-Weinberg equilibrium [HWE]), and offspring received only one Ala56 allele. The other non-transmitted 56Ala allele was present in the mother of the family paternally-transmitting the Phe465Leu variant. Of the three homozygous individuals in the 120 linked/screened families, two were affected male offspring, and one was a mother, for whom medical history information was unavailable, but who transmitted the allele to two affected males. Analysis of the unlinked multiplex and simplex families, with a 1.1% (12/1056) 56Ala frequency did not show bias in transmission (data not shown). There was a non-significant trend in the overall 384 multiplex/trio dataset for autism in males carrying one or two 56Ala alleles compared to females (15:13 affected:unaffected males vs. 5:13 affected: unaffected females; P=0.077).

Clinical Correlations. To explore phenotypic correlates with the coding variants, the inventors compared scores for trait-subsets of autism based on Autism Diagnostic Interview-Revised (ADI-R)-derived variable clusters (Table 3) previously identified from a principal components analysis of ADI-R items (Tadevosyan-Leyfer et al., 2003). These clusters reflect: (1) language, (2) social intent, (3) developmental milestones, (4) savant skills, (5) rigid-compulsive and (6) sensory aversion aspects of the autism phenotype. There was a significant increase in rigid-compulsive behaviors associated with the novel variants (P=0.0003; Table 3), and the effect was most pronounced for the Ile425Leu and Leu550Val families. The Ile425Leu substitution tracked with more severe language deficits (P=0.0031), although the brothers harboring the allele were generally more affected across most factor domains, with the exception of sensory aversions. The Leu550Val and Phe465Leu variants were associated with lesser impairment in language and social intent domains, significantly so for the Phe465Leu variant (P<0.0001 and P=0.0021, respectively). The Gly56Ala variant (heterozygous or homozygous) in the linked families similarly demonstrated a significant association with more severe rigid-compulsive behaviors (P=0.0085). When all linked families with individuals carrying a coding variant were considered together, the significance in elevated severity of rigid-compulsive behaviors increased (P=0.0002). No consistent pattern was observed across all Gly56Ala families, regardless of sex, for other ADI clusters, however, there appeared to be a trend toward two sub-groups. In one, patients had greater severity for rigid-compulsive and sensory aversion behaviors, with fewer impairments in language and social domains. The second group was absent the sensory aversion finding, but more impaired in language (Table 5).

TABLE 5
ADI Cluster Scores for SLC6A4 Coding Variant Families

| | | | | | Rigid- | Sensory |
|---|---|---|---|---|---|---|
| | Language | Social Intent | Milestones | Savant Skills | Compulsive | Aversion |
| ADI Cluster | | | | | | |
| Mean | 0.457 ± 0.268 | 0.439 ± 0.228 | 0.738 ± 0.068 | 0.124 ± 0.150 | 0.288 ± 0.209 | 0.331 ± 0.269 |
| Cases | n = 770 | n = 770 | n = 758 | n = 730 | n = 747 | n = 708 |
| Ile425Leu | *0.838 | *0.605 | 0.809 | 0.000 | 0.402 | 0.060 |
| Leu55OVal | 0.314 | 0.325 | 0.740 | 0.083 | 0.428 | 0.245 |
| Phe465Leu | **0.231 | **0.200 | 0.763 | 0.028 | 0.351 | 0.340 |
| Novel Means | 0.461 ± 0.329 | 0.377 ± 0.207 | 0.771 ± 0.035 | 0.037 ± 0.042 | 0.394 ± 0.039 | 0.215 ± 0.142 |
| | n.s. | n.s. | P = 0.052 | P = 0.0031 | P = 0.0003 | P = 0.08 |
| GIy56Ala Carriers | | | | | | |
| G56A-1 - Mean | 0.647 | 0.564 | 0.723 | 0.088 | 0.317 | 0.305 |
| G56A-2 - Mean | 0.378 | 0.164 | 0.734 | 0.671 | 0.412 | 0.667 |
| G56A-3 - Mean | 0.552 | 0.314 | 0.750 | 0.111 | 0.210 | 0.319 |
| G56A-4 - Mean | 0.207 | 0.454 | 0.780 | 0.033 | 0.497 | 0.667 |
| G56A-5 - Mean | 0.378 | 0.165 | 0.757 | 0.671 | 0.413 | 0.667 |
| G56A-6 - Mean | 0.436 | 0.344 | 0.759 | 0.109 | 0.606 | N/A |
| G56A Means | 0.462 ± 0.156 | 0.334 ± 0.158 | 0.742 ± 0.026 | 0.215 ± 0.238 | 0.409 ± 0.138 | 0.525 ± 0.195 |
| G56A Cases | n = 13 | n = 13 | n = 13 | n = 13 | n = 13 | n = 11 |
| | n.s. | P = 0.075 | n.s. | n.s. | P = 0.0085 | P = 0.0005 |
| Male GIyS6AIa Carriers | | | | | | |
| G56A Means | 0.541 ± 0.297 | 0.444 ± 0.203 | 0.754 ± 0.056 | 0.150 ± 0.157 | 0.343 ± 0.138 | 0.353 ± 0.272 |
| | n = 9 | n = 9 | n = 9 | n = 9 | n = 9 | n = 7 |
| | n.s. | n.s. | n.s. | n.s. | n.s. | n.s. |
| All Coding Means | 0.442 ± 0.205 | 0.370 ± 0.169 | 0.757 ± 0.026 | 0.199 ± 0.270 | 0.404 ± 0.111 | 0.409 ± 0.231 |
| | n.s. | P = 0.095 | P = 0.0069 | n.s. | P = 0.0002 | n.s. |

Note
*I425L-Language: Mean = 0.838 ± 0.034; P = 0.0031; Social Intent: Mean = 0.605 ± 0.026; P = 0.011
**F46SL-Language: Mean = 0.231 ± 0.0035; P < 0.0001; Social Intent: Mean = 0.200 ± 0.06; P = 0.0021

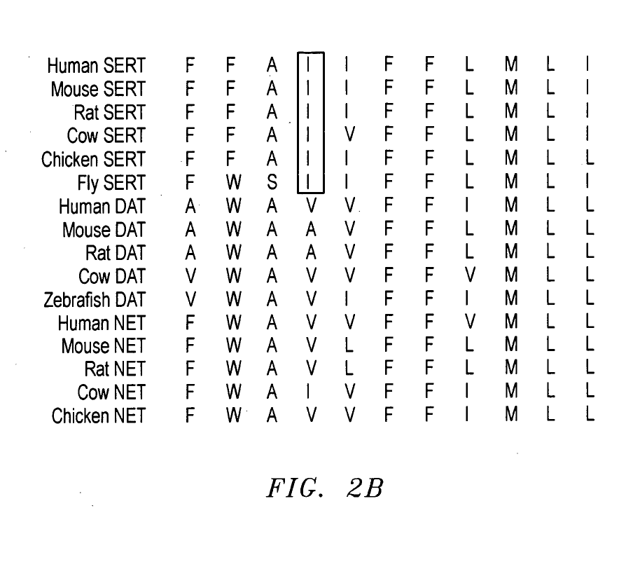

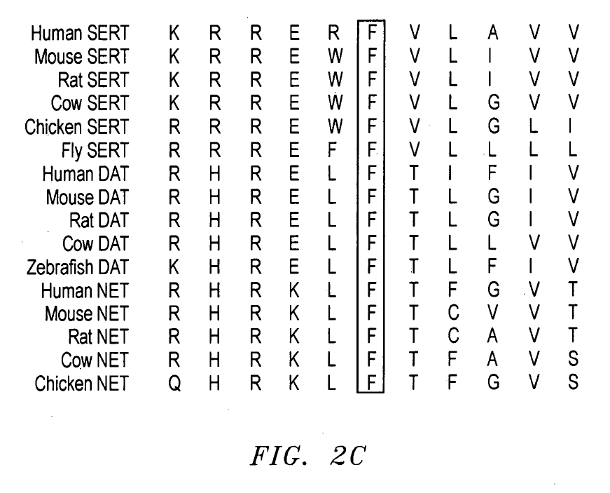

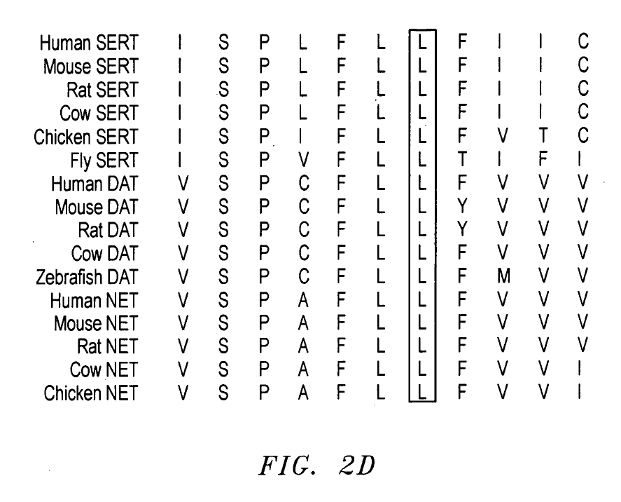

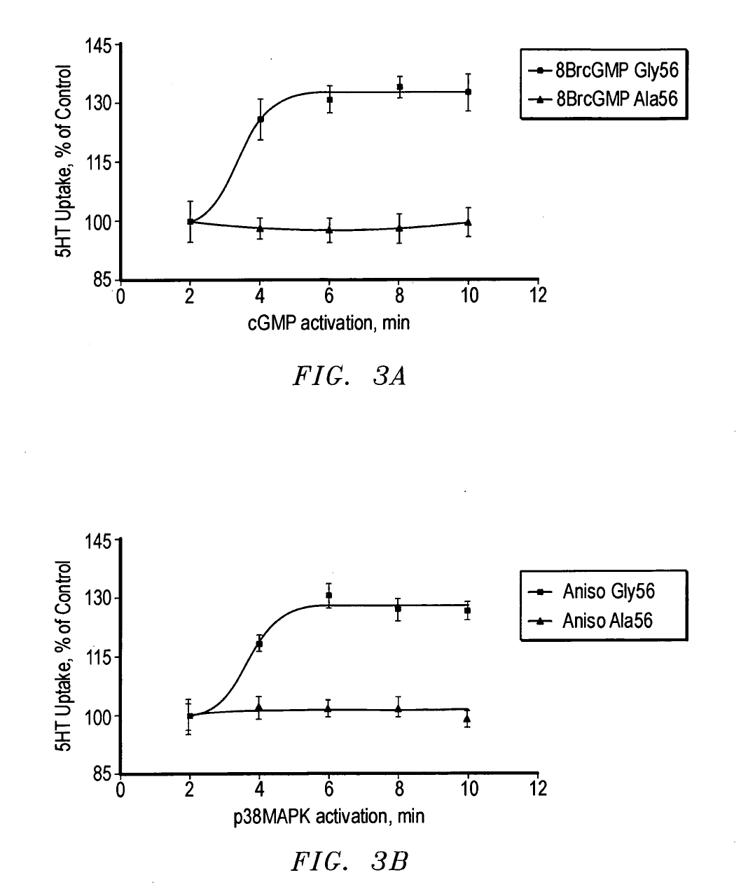

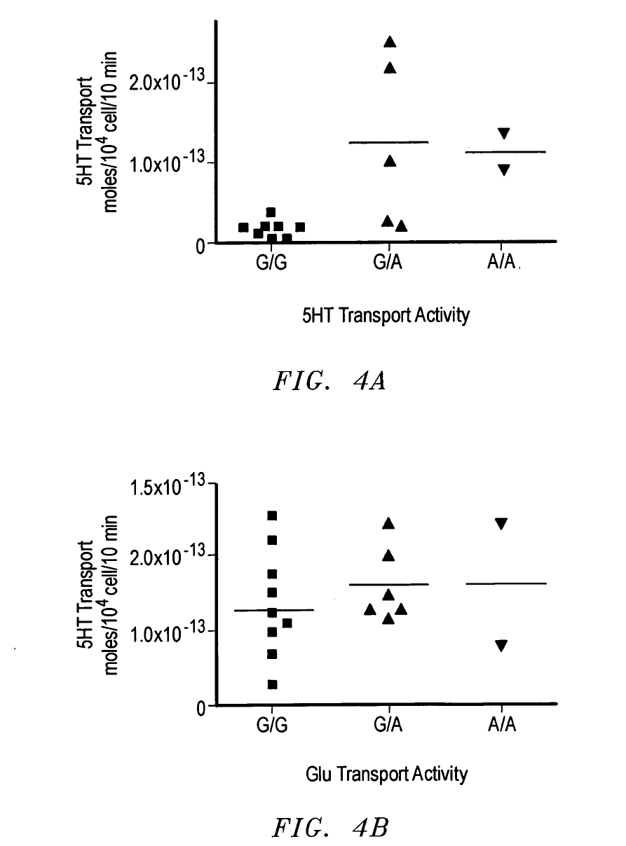

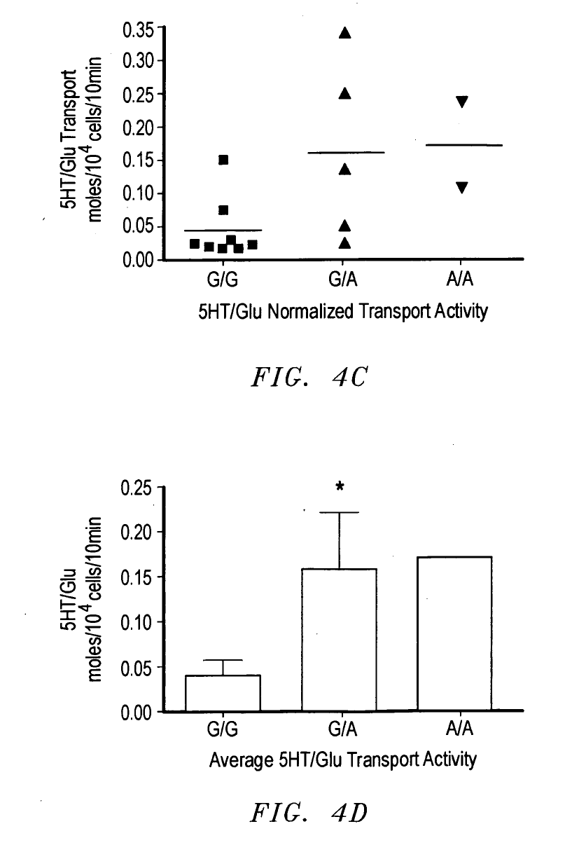

Functional Properties of Gly56Ala SERT. The presence of multiple homozygous 56Ala subjects in our multiplex autism sample allowed evaluation of the functional impact of a 56Ala substitution on SERT activity and regulation utilizing genotyped, Epstein-Barr virus (EBV)-transformed lymphocytes, as SLC6A4 is natively expressed in these cells (Khan et al., 1996; Lesch et al., 1996; Faraj et al., 1997). Basal 5-HT transport activity was elevated in Ala56-expressing cells (T-test, P<0.05), compared to either Gly56 homozygous, Gly56Ala heterozygous or combined Ala56 genotypes; significance remains after normalization to L-glutamate transport activity, not segregating with SLC6A4 genotype (FIGS. 4A-D). To control for potential confounding effects of variation in SLC6A4 gene expression and downstream basal 5-HT uptake associated with 5-HTTLPR and intron 2 VNTR genotypes (Lesch et al., 1996; Ogilvie et al., 1996; MacKenzie and Quinn 1999), the inventors repeated studies with two cell lines of each Gly56Ala genotype, carrying identical 5HTTLPR (L/L) and intron 2 VNTR (10/10) genotypes. FIG. 1F demonstrates a 56Ala dosage-dependent effect on basal 5-HT transport activity with approximately 75% increase evident for the 56Ala homozygous lines as compared to lines homozygous for 56Gly. In these cells, the inventors also demonstrated that the 56Ala allele is refractory to regulation by acute application of activators of protein kinase G or p38 mitogen activated protein kinase (Miller and Hoffman, 1994; Qian et al., 1997; Ramamoorthy and Blakely, 1999; Zhu et al., 2004; Samuvel et al., 2005; Zhu et al., 2005) (FIGS. 3A-B), suggesting that intrinsic features of function unlikely to be attributable to a linked, noncoding variant are evident. The inventors recently described a similar loss of regulation despite normal surface density for 56Ala cDNA transfected into HeLa cells (Prasad et al., submitted).

To more completely evaluate the SLC6A4 locus for novel alleles, the inventors expanded the screen to cover proximal promoter sequences and noncoding 5′ and 3′ exons. Thirty-one families were subsequently analyzed for allelic segregation patterns for rare noncoding variants discovered across the locus. Variants were identified in the promoter, exon 1b, exon 14 and intronic sequences in the linked families (Table 4). Several variants are known polymorphisms with existing database identifiers, although many are novel. Other polymorphisms with modest to high minor allele frequency were tested previously for association and are not included in Table 4. Allelic segregation patterns reveal a stark pattern of transmission disequilibrium (TD), when all variants are combined together for purposes of considering segregation. Redundant allelic transmissions represented by two or more SNPs were not counted more than once. TD was evident from a T:NT count of 76:12, representing a highly significant deviation from the null hypothesis of no association (c2=26.82; 1 df; P=2.2×10−7). There is an a priori expectation of transmission (or de novo sequence change) to index cases in whom variants were originally detected. To reduce this bias, transmissions to these individuals were excluded, yet segregation remains significant (T:NT=40:12, c2=8.13; 1 df; P=0.0042). Therefore, these multiple coding and noncoding alleles persist in demonstrating a collective linkage to and allelic segregation with autism, despite failing to identify association of common alleles (apart from HTTLPR and rs140700) at SLC6A4 in these families.

### EXAMPLE 3

### Comparative Results for Each of Four Mutants

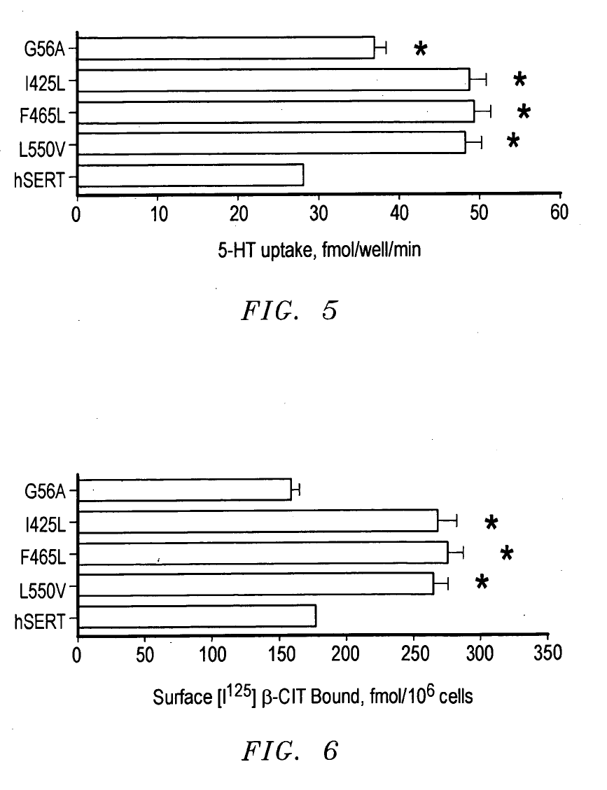

Transport Assays of Autism-Associated SERT Coding Variants in Transfected Cells. To initiate an analysis of autism-associated SERT coding variants in transfected cells, the inventors used site-directed mutagenesis to engineer the alleles Ile425Leu, Phe465Leu, and Leu550Val into hSERT mammalian expression constructs as described above with Gly56Ala. By fluorescence dideoxynucleotide-based sequencing of both strands of our constructs, the inventors verified that we have achieved the intended hSERT mutations and no others. The inventors performed initial transfection studies in HeLa cells, monitoring [³H]5HT transport activity in hSERT or the hSERT mutant alleles 2 days after transfection. As there is evidence of the properties of SERT changing with overexpression, in prior control studies, they developed expression conditions so as to match hSERT expression levels to those found in native human lymphocytes (data not shown). As shown in FIG. 5, each of the autism-associated variants tested at 20 nM [³H]5HT displays significantly elevated transport activity, with highest levels seen for Ile425Leu, Phe465Leu and Leu550Val. These studies will be further replicated and extended to achieve a full kinetic analysis of the impact of mutations on transport kinetics, determining 5 HT Km and Vmax as well as sensitivity to blockade by antagonists. Below, the inventors note their efforts to insure that these changes reflect behaviors of modified SERT when expression is derived from the native hSERT gene (lymphoblast studies) as well as to monitor expression in neurons (transgenic mouse studies) most appropriate for association with autism traits.

Analysis of Surface Density of Autism-Associated SERT Coding Variants in Transfected Cells. As they had preliminary findings that the autism-associated SERT coding variants were gain-of-function alleles, the inventors next proceeded to assess whether these increases arise from changes in SERT trafficking or catalytic activity. In our prior study of a large number of published or deposited human SERT coding variants unassociated (at the time) with a clinical phenotype, they found cases where uptake activity enhancements were due to changes in catalytic rates (Gly56Ala) on the one hand and elevated trafficking (e.g., Ile425Val) on the other. They also identified alleles where significantly reduced activity was associated with reduced surface trafficking (e.g., Pro339Leu). As shown in FIG. 6, examination of 5HT-sensitive [1²⁵]RTI-55 binding reveals that of the autism-associated SERT alleles, Ile425Leu, Phe465Leu and Leu550Val demonstrate elevated surface binding relative to hSERT or to Gly56Ala. These studies indicate that elevations in three of the autism-associated alleles is due to enhanced constitutive surface trafficking whereas Gly56Ala achieves elevated activity, as noted previously, through changes in catalytic function. These data indicate that elevated function may be a final common impact of different mechanisms impacted by autism-associated hSERT alleles.

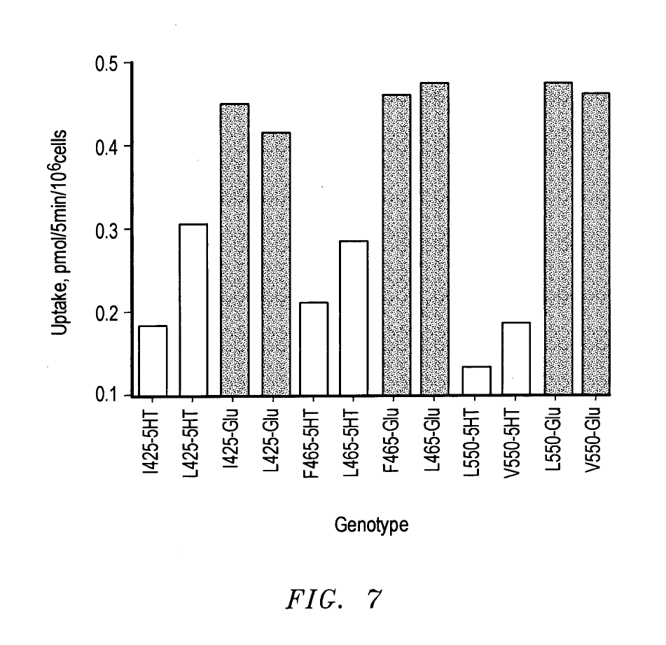

Measurement of SERT Activity in Lymphoblasts from Autism Subjects. Transfection experiments suggest that autism-associated SERT mutations are gain-of-function alleles. Certainly, this is an artificial paradigm and must be extended to natively expressed SERT proteins and ultimately to the brain. Fortunately, SERTs are expressed in lymphocytes and lymphocyte lines from which DNA samples have been extracted. The inventors obtained single lines carrying the Ile425Leu, Phe465Leu, and Leu550Val variants (all are heterozygotes) as well as 5HTTLPR and intron 2 VNTR matched control lymphocytes from family members. In FIG. 7, they show 5HT transport assays conducted with these lines where they can demonstrate that, in each case, the mutant allele confers an elevation of SERT activity. To control for these measurements and insure they do not arise from nonspecific changes in ion gradients supporting transport, the inventors assessed [³H]glutamate transport in parallel. As can be seen in FIG. 7, [³H]glutamate transport in these mutant lines is equivalent to or below the level of that found in the lines carrying non-mutant SERTs, suggesting that the 5HT transport elevations can be specifically associated with the SERT alleles. Future studies will replicate these experiments and add additional lines to the analysis as well as gather data on SERT surface density using whole cell radioligand binding assays and monitor native regulatory potential through PKG, PKC and p38 MAPK pathways.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 3,817,837
U.S. Pat. No. 3,850,752
U.S. Pat. No. 3,939,350
U.S. Pat. No. 3,996,345
U.S. Pat. No. 4,275,149
U.S. Pat. No. 4,277,437
U.S. Pat. No. 4,366,241
U.S. Pat. No. 4,659,774
U.S. Pat. No. 4,683,195
U.S. Pat. No. 4,683,202
U.S. Pat. No. 4,800,159
U.S. Pat. No. 4,816,571
U.S. Pat. No. 4,959,463
U.S. Pat. No. 5,141,813
U.S. Pat. No. 5,242,974
U.S. Pat. No. 5,264,566
U.S. Pat. No. 5,384,261
U.S. Pat. No. 5,405,783
U.S. Pat. No. 5,412,087
U.S. Pat. No. 5,424,186
U.S. Pat. No. 5,428,148
U.S. Pat. No. 5,429,807
U.S. Pat. No. 5,436,327
U.S. Pat. No. 5,445,934
U.S. Pat. No. 5,472,672
U.S. Pat. No. 5,527,681
U.S. Pat. No. 5,529,756
U.S. Pat. No. 5,532,128
U.S. Pat. No. 5,545,531
U.S. Pat. No. 5,554,501
U.S. Pat. No. 5,554,744
U.S. Pat. No. 5,556,752
U.S. Pat. No. 5,561,071
U.S. Pat. No. 5,571,639
U.S. Pat. No. 5,574,146
U.S. Pat. No. 5,593,839
U.S. Pat. No. 5,599,695
U.S. Pat. No. 5,602,244
U.S. Pat. No. 5,624,711
U.S. Pat. No. 5,658,734
U.S. Pat. No. 5,700,637
U.S. Pat. No. 5,757,994
U.S. Pat. No. 5,788,166
U.S. Pat. No. 5,838,002
U.S. Pat. No. 5,840,873
U.S. Pat. No. 5,843,640
U.S. Pat. No. 5,843,650
U.S. Pat. No. 5,843,651
U.S. Pat. No. 5,843,663
U.S. Pat. No. 5,846,708
U.S. Pat. No. 5,846,709
U.S. Pat. No. 5,846,717
U.S. Pat. No. 5,846,726
U.S. Pat. No. 5,846,729
U.S. Pat. No. 5,846,783
U.S. Pat. No. 5,849,481
U.S. Pat. No. 5,849,486
U.S. Pat. No. 5,849,487
U.S. Pat. No. 5,849,497
U.S. Pat. No. 5,849,546
U.S. Pat. No. 5,849,547
U.S. Pat. No. 5,851,772
U.S. Pat. No. 5,853,990
U.S. Pat. No. 5,853,992
U.S. Pat. No. 5,853,993
U.S. Pat. No. 5,856,092
U.S. Pat. No. 5,858,652
U.S. Pat. No. 5,861,244
U.S. Pat. No. 5,863,732
U.S. Pat. No. 5,863,753
U.S. Pat. No. 5,866,331
U.S. Pat. No. 5,866,366
U.S. Pat. No. 5,882,864
U.S. Pat. No. 5,900,481
U.S. Pat. No. 5,905,024
U.S. Pat. No. 5,910,407
U.S. Pat. No. 5,912,124
U.S. Pat. No. 5,912,145
U.S. Pat. No. 5,912,148
U.S. Pat. No. 5,916,776
U.S. Pat. No. 5,916,779
U.S. Pat. No. 5,919,626
U.S. Pat. No. 5,919,630
U.S. Pat. No. 5,922,574
U.S. Pat. No. 5,925,517
U.S. Pat. No. 5,928,862
U.S. Pat. No. 5,928,869
U.S. Pat. No. 5,928,905
U.S. Pat. No. 5,928,906
U.S. Pat. No. 5,929,227
U.S. Pat. No. 5,932,413
U.S. Pat. No. 5,932,451
U.S. Pat. No. 5,935,791
U.S. Pat. No. 5,935,825
U.S. Pat. No. 5,939,291
U.S. Pat. No. 5,942,391
U.S. Pat. No. 5,986,258
U.S. Pat. No. 6,004,755
U.S. Pat. RE 35,413
U.S. Ser. No. 10/407,846
Abbondanzo, Ann. Diagn. Pathol., 3(5):318-327, 1999.
Allred et al., Arch. Surg., 125(1):107-113, 1990.
Antibodies: A Laboratory Manual, Cold Spring Harbor Laboratory, Cold Spring Harbor Press, Cold Spring Harbor, N.Y., 1988.
Ashley-Koch et al., Genomics, 61:227-236, 1999.
Auranen et al., Mol. Psychiatry., 5:320-322, 2000.
Bahr et al., J. Mass Spectrom., 32:1111-1116, 1997.
Barnes et al., J. Pharm. Pharmacol., 40(9):668, 1988.
Beaucage, Methods Mol. Biol., 20:33-61, 1993.
Bentzley et al., Anal Chem., 68(13):2141-2146, 1996.
Blackman et al., Biophys. J., 71(1):194-208, 1996.
Blundell, Appetite, 7:39-56, 1986.
Bolton et al., Psychol Med., 28:385-395, 1998.
Briley and Moret, Trends Pharmacol Sci., 14(11):396-397, 1993.
Brown et al., Immunol Ser, 53:69-82, 1990.
Bucknall et al., J. Am. Soc. Mass Spectrom., 13(9):1015-1027, 2002.
Buxbaum et al., Am. J. Hum. Genet., 68:1514-1520, 2001.
Buxbaum et al., Mol. Psychiatry, 9:144-150, 2004.
Cantor et al., Am. J. Hum. Genet., 76, 2005.
Caprioli et al., Anal. Chem., 69:4751, 1997.
Cargill et al., Nat. Genet., 22:231-238, 1999.
Chakrabarti and Fombonne, JAMA, 285:3093-3099, 2001.
Chaurand et al., Anal Chem., 71(23):5263-5270, 1999.
Chen et al., Nat. Biotechnol., 19:537-542, 2001.
Collaborative Linkage Study of Autism, Am. J. Med. Genet., 105B:539-547, 2001.
Collaborative Linkage Study of Autism, Am. J. Med. Genet., 88B:609-615, 1999.
Conroy et al., Mol. Psychiatry., 9:587-593, 2004.
Cook and Leventhal, Curr. Opin. Pediatr., 8:348-354, 1996.
Cook et al., Life Sciences, 52:2005-2015, 1993.
Cook et al., Mol. Psychiatry, 2:247-250, 1997.
De Jager et al., Semin. Nucl. Med., 23(2):165-179, 1993.
den Dunnen and Antonarakis, Hum. Genet., 109:121-124, 2001.
Desiderio et al., J. Mass Spectrom., 35(6):725-733, 2000.
Desiderio et al., Methods Mol. Biol., 61:57-65, 1996.
Doolittle and Ben-Zeev, Methods Mol Biol, 109:215-237, 1999.
Duncan et al., Rapid Commun. Mass Spectrom., 7(12): 1090-1094, 1993.
European Application No. 320 308
Faraj et al., Biochem. Pharmacol., 53:53-57, 1997.
Faulstich et al., Anal. Chem., 69(21):4349-4353, 1997.
Folstein and Rosen-Sheidley, Nat. Rev. Genet., 2:943-955, 2001.
Folstein and Rutter, Nature, 265:726-728, 1977.
Fombonne, JAMA, 289:87-89, 2003.
Frohman, In: PCR Protocols: A Guide To Methods And Applications, Academic Press, N.Y., 1990.
GB Appln. 2 202 328
Gillam et al., J. Biol. Chem., 253(8):2532-2539, 1978.
Glatt et al., Nat. Genet., 27:435-438, 2001.
Gobom et al., Anal. Chem., 72(14):3320-3326, 2000.
Gobom et al., Anal. Chem., 72(14):3320-3326, 2000.
Gordon et al, Genome Res., 11:614-625, 2001
Gu et al., J. Biol. Chem., 269:7124-7130, 1994.
Gudbjartsson et al., Nat. Genet., 25:12-13, 2000.
Guilfoyle et al., Nucleic Acids Research, 25:1854-1858, 1997.
Gulbis and Galand, Hum. Pathol., 24(12):1271-1285, 1993.
Hahn and Blakely, Pharmacogenomics J, 2:217-235, 2002.
Hollander et al., Neuropsychopharmacology, 30:582-589, 2005.
Holloway et al., Hum. Mutat., 14:340-347, 1999.
Horak et al., Rapid Commun. Mass Spectrom., 15(4):241-248, 2001.
Innis et al., Proc. Natl. Acad. Sci. USA, 85(24):9436-9440, 1988.
International Molecular Genetic Study of Autism Consortium, Am. J. Hum. Genet., 69:570-581, 2001
International Molecular Genetic Study of Autism Consortium, Hum. Mol. Genet., 7:571-578, 1998.
International Molecular Genetic Study of Autism Consortium, Hum. Mol. Genet., 10:973-982, 2001.
Itakura et al., J. Am. Chem. Soc., 97(25):7327-7332, 1975.
Jespersen et al., Anal Chem., 71(3):660-666, 1999.
Jiang et al., J. Agric. Food Chem., 48:3305, 2000.
Jones and Szatmari, J. Autism Dev. Disord., 18:31-40, 1988.
Kabarle et al., Anal. Chem. 65(20):972A-986A, 1993.
Kanazawa et al., Biol. Pharm. Bull., 22(4):339-346, 1999.
Kanner, Nervous Child, 2:217-250, 1943.
Kazmaier et al., Anesthesiology, 89(4):831-817, 1998.
Khan et al., Cell Immunol., 172:269-274, 1996.
Khorana, Science, 203(4381):614-625, 1979.
Kilic et al., Mol. Pharmacol., 64:440-446, 2003.
Kim et al., Mol. Psychiatry, 7:278-288, 2002.
Klauck et al., Hum. Mol. Genet., 6:2233-2238, 1997.
Kong et al., Nat. Genet., 31:241-247, 2002.
Kwoh et al., Proc. Natl. Acad. Sci. USA, 86: 1173, 1989.
Le Bars et al., Pain, 32(2):259-261, 1988.
Lesch et al., Science, 274:1527-1531, 1996.
Li et al., Electrophoresis, 23:1499-1511, 2002.
Li et al., Trends Biotechnol., 18:151, 2000.
Liu et al., Am. J. Hum. Genet., 69:327-340, 2001.
Lovelace et al., J. Chromatogr., 562(1-2):573-584, 1991.
Lynn et al., J. Mol. Evol., 48(5):605-614, 1999.
MacKenzie and Quinn, Proc. Natl. Acad. Sci. USA, 96:15251-15255, 1999.
Maestrini et al., Am. J. Med. Genet., 88B:492-496, 1999.
Marie et al., Anal. Chem., 72(20):5106-5114, 2000.
Martin et al., Am. J. Hum. Genet., 67:146-154, 2000.
McCauley et al., Am. J. Hum. Genet., 127B: 104-112, 2004.
McCauley et al., BMC Med. Genet., 6:1, 2005.
McPeek, Genet. Epidemiol., 16:225-249, 1999.
Meltzer et al., Arch. Gen. Psychiatry, 38(12):1322-1326, 1981.
Miketova et al., Mol. Biotechnol., 8(3):249-253, 1997.
Miller and Hoffman, J. Biol. Chem., 269:27351-27356, 1994.
Mirgorodskaya et al., Rapid Commun. Mass Spectrom., 14(14):1226-1232, 2000.
Muddiman et al., Fres. J. Anal. Chem., 354:103, 1996.
Nakamura et al., In: Handbook of Experimental Immunology (4^{th} Ed.), Weir et al., (eds). 1:27, Blackwell Scientific Publ., Oxford, 1987.
Naranjo et al., Clin. Pharmacol. Ther., 41(3):266-274, 1987.
Nelson et al., Anal. Chem., 66:1408, 1994.
Neumeister et al., Arch. Gen. Psychiatry, 59(7):613-620, 2002.
Nguyen et al., J. Chromatogr. A., 705(1):21-45, 1995.
Nickerson et al., Methods Mol. Biol., 175:29-35, 2001.
Nickerson et al., Nucleic Acids Res., 25:2745-2751, 1997.
Ogilvie et al., Lancet., 347:731-733, 1996.
Ohara et al., Proc. Natl. Acad. Sci. USA, 86: 5673-5677, 1989.
Ozaki et al., Mol. Psychiatry, 8:895, 933-896, 2003.
Pagani and Baralle, Nat. Rev. Genet., 5:389-396, 2004.
U.S. Pat. No. 4,883,750
Paul et al., Arch. Gen. Psychiatry, 38(12):1315-1317, 1981.
PCT Appln. PCT/US87/00880
PCT Appln. PCT/US89/01025
PCT Appln. WO 84/03564
PCT Appln. WO 88/10315
PCT Appln. WO 89/06700
PCT Appln. WO 90/07641
Perry et al., Br. J. Psychiatry, 142:188-192, 1983.
Persico et al., Am. J. Med. Genet., 96B:123-127, 2000.
Philippe et al., Hum. Mol. Genet., 8:805-812, 1999.
Pickles et al., Am. J. Med. Genet., 57:717-726, 1995.
Qian et al, J. Neurosci., 17:45-57, 1997.
Ramamoorthy and Blakely, Science, 285:763-766, 1999.
Ramamoorthy et al., Biochemistry, 32:1346-1353, 1993.
Risch et al., Am. J. Hum. Genet., 65:493-507, 1999.
Ritvo et al., Am. J. Psychiatry, 146:194-199, 1989.
Roepstorff, EXS., 88:81-97, 2000.
Sambrook et al., In. Molecular cloning, Cold Spring Harbor Laboratory Press, Cold Spring Harbor, N.Y., 1989.
Samuvel et al., J. Neurosci., 25:29-41, 2005.
Schain and Freedman, J. Pediatr., 58:315-320, 1961.
Schloss et al., J. Psychopharmacol., 12(2):115-21, 1998.
Shao et al., Am. J. Hum. Genet., 70:1058-1061, 2002a.
Shao et al., Am. J. Med. Genet., 114B:99-105, 2002b.
Silverstone et al., Appetite, 7::85-97, 1986.
Soubrie et al., Brain Res., 437(2):323-331, 1987.
Spielman and Ewens, Am. J. Hum. Genet., 59:983-989, 1996.
Stoeckli et al., Nat. Med., 7(4):493-496, 2001.
Stone et al., Am. J. Hum. Genet., 75:1117-1123, 2004.
Suranyi-Cadotte et al., Life Sci., 36(8):795-799, 1985.
Tadevosyan-Leyfer et al., J. Am. Acad. Child Adolesc. Psychiatry, 42:864-872, 2003.
Takach et al., J. Protein Chem., 16:363, 1997.
Tordjman et al., Mol. Psychiatry, 6:434-439, 2001.
Tsai et al., Neuropsychobiology, 45(3):131-133, 2002.
Van Woert et al., Monogr. Neural. Sci., 3:71-80, 1976.
Veenstra-VanderWeele and Anderson, Eur. J. Pharmacol., 410:165-181, 2000.
Veenstra-Vanderweele et al., Annu. Rev. Genomics Hum. Genet., 5:379-405, 2004.
Villanueva et al., Enzyme Microb. Technol., 29:99, 1999.
Walker et al., Proc. Natl. Acad. Sci. USA, 89:392-396 1992.
Wang et al., Anal. Chem., 72(21):5285-5289, 2000.
Wang et al., J. Agric. Food. Chem., 47:1549, 1999.
Wang et al., J. Agric. Food. Chem., 47:2009, 1999.
Weiss et al., Am. J. Hum. Genet., 76:33-41, 2005.
Wittmann et al., Biotechnol. Bioeng., 72:642, 2001.
Wu et al., Anal. Chem., 70:456A, 1998.
Wu et al., Biochem. Biophys. Res. Commun., 233(1):221-226, 1997.
Wu et al., Biochim. Biophys. Acta, 1466:315-327, 2000.
Yang et al., J. Agric. Food. Chem., 48:3990, 2000.
Yeargin-Allsopp et al., JAMA, 289:49-55, 2003.
Yirmiya et al., Am. J. Med. Genet., 105B:381-386, 2001.
Yonan et al., Am. J. Hum. Genet., 73:886-897, 2003.
Zhong et al., Clin. Chem. ACTA., 313:147, 2001.
Zhu et al., J. Biol. Chem., 280:15649-15658, 2005.
Zhu et al., Mol. Pharmacol., 65:1462-1474, 2004.
Zweigenbaum et al., Anal. Chem., 71(13):2294-300, 1999.
Zweigenbaum et al., J. Pharm. Biomed. Anal., 23(4):723-733, 2000.

What is claimed is:

1. A method of identifying a subject having or at risk of developing autism, an austism spectrum disorder or an associated disorder comprising assessing the expression or mutation of a gene located at SLC6A4.

2. The method of claim 1, wherein said subject exhibits one or more clinical symptoms of autism.

3. The method of claim 1, wherein said subject does not exhibit a clinical symptom of autism.

4. The method of claim 1, wherein said subject has previously been diagnosed with autism.

5. The method of claim 1, wherein said subject has a family member that has previously been diagnosed with autism.

6. The method of claim 1, wherein said subject has not previously been diagnosed with autism.

7. The method of claim 1, wherein assessing comprises measuring the expression level of SERT protein.

8. The method of claim 1, wherein assessing comprises determining the structure of SERT protein.

9. The method of claim 1, wherein assessing comprises measuring the expression level or structure of a SLC6A4 transcript.

10. The method of claim 9, wherein measuring comprises Northern blot or quantitative RT-PCR of SLC6A4.

11. The method of claim 1, wherein assessing comprises determining the structure of a SLC6A4 gene.

12. The method of claim 11, wherein assessing comprises determining the structure of a SLC6A4 coding region.

13. The method of claim 11, wherein assessing comprises determining the structure of a SLC6A4 non-coding region.

14. The method of claim 13, wherein said non-coding region is a promoter, intron or 3′ non-coding region.

15. The method of claim 11, wherein assessing comprises sequencing, primer extension, restriction digestion, SNP specific oligonucleotide hybridization, or DNAse protection.

16. The method of claim 1, wherein assessing a mutation comprises identifying at change in SLC6A4 exon 1b, exon 2, exon 9, exon, 10, exon 12, exon 13, exon 14, intron 1a, intron 1b, intron 6, intron 7, intron 8, or the SLC6A4 promoter.

17. The method of claim 1, wherein said subject is a male.

18. The method of claim 1, further comprising making a treatment decision based on the result of assessing.

19. The method of claim 1, wherein said subject exhibits one or more of Gly56Ala, Ile425Leu, Phe465Leu and Leu550Val.

20. The method of claim 1, wherein said subject exhibits two or more of Gly56Ala, Ile425Leu, Phe465Leu and Leu550Val.

21. A method of identifying a subject having or at risk of developing autism comprising assessing the subject's SERT activity.

22. The method of claim 21, further comprising obtaining a tissue sample from said subject.

23. The method of claim 21, wherein said subject exhibits one or more clinical symptoms of autism.

24. The method of claim 21, wherein said subject does not exhibit a clinical symptom of autism.

25. The method of claim 21, wherein said subject has previously been diagnosed with autism.

26. The method of claim 21, wherein said subject has a family member that has previously been diagnosed with autism.

27. The method of claim 21, wherein said subject has not previously been diagnosed with autism.

28. The method of claim 21, wherein said subject is a male.

29. The method of claim 21, further comprising making a treatment decision based on the result of assessing.

30. A nucleic acid primer for amplification of a SLC6A4 gene at a position selected from the group consisting 425, 465 and 550 of SEQ ID NO:1.

31. A nucleic acid probe that selectively hybridizes to a SLC6A4 gene encoding 425Leu, 465Leu or 550Val of SEQ ID NO:2.

32. An antibody that binds to a SERT protein having 425Leu, but that does not bind to a SERT protein have 42511e.

33. An antibody that binds to a SERT protein having 465Leu, but that does not bind to a SERT protein have 425Phe.

34. An antibody that binds to a SERT protein having 550Val, but that does not bind to a SERT protein have 550Leu.

35. A method of screening for an agent that can modulate one or more symptoms of autism, an autism spectrum disorder or an associated disorder comprising (a) contacting a cell that expresses SERT with said agent; (b) measuring SERT activity; and (c) comparing the SERT activity observed in (b) with that seen in the absence of said agent, whereby a difference in the activity observed in (b) and (c) indicates that said agent modulates one or more symptoms of autism.

36. The method of claim 35, wherein said agent inhibits SERT activity.

37. The method of claim 35, wherein said agent increases SERT activity.

38. The method of claim 36, wherein said agent is an antisense SERT nucleic acid, a SERT siRNA or a SERT-binding antibody.

39. The method of claim 37, wherein said agent is a SERT-encoding expression construct.
